SEQUENCE LISTING (12 sequences)

SEQ ID NO: 1 - protein, 219 aa, Artificial Sequence
  Synthetically generated peptide
VLVDEPNVTEVLIRVHRRKILLKNPWTKEEHQVVWSEWSTTCGTATRKRWTECSATCGGGTKHRERWTEYSACSRTCGGGTQERKRSYYNSPYYSYSSYPSYYNYSYPSYSYSSYPSYYRYSSYPYYNYSYPSYYNYGSYPYYSYSSYPSWYGAGVAGLQAISTAHGLGAQVFGHDVRSATREEVESCGGKFIGLRMGEEAEVLGGYAREMGDAYQRAQ

SEQ ID NO: 2 - protein, 129 aa, Artificial Sequence
  Synthetically generated peptide
SPPSTPVSPPSTPVSPPSTPVSPPSTPVVSVTEPNVDEVLIQIRNKQIFLKNPWTGQEEQVLVLVDEPNVTEVLIRVHRRKILLKNPWTKEEHQVVVVVVVVVGSSMHVVEVRSFGVRRRPSTESRRSS

SEQ ID NO: 3 - protein, 349 aa, Artificial Sequence
  Synthetically generated peptide
VLVDEPNVTEVLIRVHRRKILLKNPWTKEEHQVVVVVVVVVGSSMHVVEVRSFGVRRRPSTESRRSSSCAVRGSRYGTIPISTQTVDNATLCQQQCQKSSMCEAFSYDIKGKVCYLHVAYAAKLKRANYNFISGPRQCAWSEWSTTCGTATRKRWTECSATCGGGTKHRERWTEYSACSRTCGGGTQERKRWSTTCGSATRQRVWSDWSDCSATCGGGTRYRERWTEFSDCSRVCGGGTKERRRWTACGDPSEGLRTRTRWSECKNGKQYRGAAGCASVYEVRACSGASDAKECWSPWTICRDGMQTRDCKSLGVQESRPCSAEGEAPKGEGGQEKPSVPLIAVRIHGS

SEQ ID NO: 4 - protein, 269 aa, Artificial Sequence
  Synthetically generated peptide
VLVDEPNVTEVLIRVHRRKILLKNPWTKEEHQVVVVVVVVVGSSMHVVEVRSFGVRRRPSTESRRSSKKKKVMYMSQPMKVAPPPMKYAPPTKAHPVMMAAPAKMAPAPMIVEAAPMKKGRYLAAEDETEMVFEAENFQTEQYDSAPERSLGKKKVMYVSEPVKMASPPVMMAAPTKMSAPMVVRAAPTKAPAPMIVQAAPTKSYYNSPYYSYSSYPSYYNYSYPSYSYSSYPSYYRYSSYPYYNYSYPSYYNYGSYPYYSYSSYPSWY

SEQ ID NO: 5 - protein, 219 aa, Artificial Sequence
  Synthetically generated peptide
VLVDEPNVTEVLIRVHRRKILLKNPWTKEEHQVVLLNYHNSQYFGEIKIGTPGRRFVVVFDTGSSNLWVPAAEGAGVAGLQAISTAHGLGAQVFGHDVRSATREEVESCGGKFIGLRMGEEAEVLGGYAREMGDAYQRAQQSQVLLRVSAPSPDLVSRIPRDKVLISYLFPSINQQALDDLEQSLEAGKQGAECLLRSSKLALEALLEGARVAATRGLL

SEQ ID NO: 6 - DNA, 676 bp, Artificial Sequence
  Synthetically generated oligonucleotide
tctagaaatggtgctcgtcgatgagcctaatgtcaccgaagttttgattcgtgtgcaccgtagaaagattctcctgaaaaacccatggacgaaggaggaacatcaagtcgtctggagcgagtggagcaccacttgcggcaccgctactcgcaaacgttggaccgagtgttccgccacttgcgggggtggaaccaagcacagggagaggtggaccgagtactctgcctgctctcgcacttgtggtgggggaacacaagaaagaaaacgctcgtattataactcaccttattatagctatagctcttatccaagttattacaattactcctatccgagctactcctattcatcgtatccttcgtattatcgatattcgtcctacccatactacaactattcctatccctcctactacaactatggttcgtatccttattactcgtactctagctacccaagttggtacggcgccggcgtggcagggctgcaagctatctcaactgctcatggccttggggcccaggtttttggccacgacgttcgttcagcaacaagggaggaggtagaaagttgtggtggtaagttcatcggtctaagaatgggtgaagaggcggaagtgttaggaggatatgccagggaaatgggcgacgcgtatcagcgggctcaatgactcgag

SEQ ID NO: 7 - DNA, 412 bp, Artificial Sequence
  Synthetically generated oligonucleotide
tctagaaatgtcgcctccatccactccggtcagcccaccatctaccccagtcagccccccctcaacacctgtatctcctccgagcaccccagtggtcagcgttactgaacctaatgtcgatgaggttttgatacaaattcgtaataagcaaatctttctaaaaaacccatggactggccaagaggagcaagtccttgtgctcgtggacgaacctaacgtgacggaagtattaattcgagtccataggagaaaaatccttctgaagaacccatggaccaaggaggagcaccaggttgtggtggtggttgttgtggtagtcggttcctcaatgcacgtggttgaagtcaggtctttcggagtccgcaggcgcccatctacagaatcccggagaagcagttgactcgagggtacc

SEQ ID NO: 8 - DNA, 1072 bp, Artificial Sequence
  Synthetically generated oligonucleotide
tctagaaatggttcttgtggatgaacctaatgttaccgaggtgttaattagagttcacaggcgcaagatcttgctaaaaaacccctggacgaaggaggagcaccaggttgtggtggttgttgtcgtcgtcgttggttcatctatgcatgtagtagaagtgagatcattcggggttcgaagacggccgtccacagagagtagacgttcgtcatcgtgcgctgtgcgtggtagccgttacggaactataccgatttctacccagacggtggacaacgcaacactgtgtcagcagcaatgtcaaaaatcttccatgtgtgaagcattcagctacgatattaagggcaaagtttgctatctccacgtcgcttatgcagccaagcttaagagggccaactacaatttcatctctggaccaaggcaatgtgcatggagcgaatggagtactacctgcggaaccgctacacgaaaaaggtggacagaatgcagtgctacatgcggaggcggaactaaacatcgtgaaaggtggactgaatactccgcctgcagcagaacatgcggaggaggaactcaagagcgtaaacgttggtcaaccacatgcggttcagccacgcgtcagcgcgtatggtccgactggtctgactgcagcgctacgtgcggtggagggaccagataccgtgaaaggtggactgagttttccgattgctccagagtttgtggaggcggaactaaggagagaaggcgctggaccgcatgtggagacccctcagagggacttcgaacgcgaacgcgatggtctgaatgtaagaacggtaagcaatacaggggggctgccggttgcgcttccgtctatgaggtacgtgcttgctctggtgctagcgacgcgaaagaatgttggagcccatggactatctgtcgggatgggatgcaaacaagagattgtaaaagcctcggtgttcaggagtcacgcccatgttcagcagagggtgaagcaccaaagggtgagggcggacaagagaaaccctcagtgccactgatcgctgtgcgaatacatggtagttgactcgagggtacc

SEQ ID NO: 9 - DNA, 832 bp, Artificial Sequence
  Synthetically generated oligonucleotide
tctagaaatggtcctcgtagacgaacccaacgtcactgaggttctaataagggttcaccgaagaaaaatccttctcaagaacccatggaccaaagaggagcatcaagtagtcgttgtcgtggtggtggtggtgggttcttcgatgcatgtggtagaggtccgtagcttcggagtcagacgtcggccttcaactgagtccaggcgcagtagcaagaagaaaaaggtgatgtacatgagtcagccaatgaaggttgccccacctcctatgaagtatgcaccgccaaccaaggcgcaccccgttatgatggcagcgccggccaagatggcccccgcccctatgattgttgaggccgcgccgatgaagaaggggaggtacttggctgctgaagatgaaacggagatggtcttcgaggccgaaaactttcaaaccgaacagtacgactcggctccagagcggtccctgggcaagaagaaggtcatgtacgttagcgaaccggttaagatggcgtctccaccagtgatgatggcagcacctaccaagatgtccgccccaatggtagttcgcgctgctcccaccaaggccccggctccaatgattgttcaggcagccccgacgaagtcttattacaattctccatattacagctactcctcatacccatcatactacaactacagctacccctcgtattcctattcctcgtatccttcatactatcgctactcctcttatccgtattacaattactcgtacccctcttactataattacggcagctacccatactactcttacagtagctatccatcttggtattgactcgagggtacc

SEQ ID NO: 10 - DNA, 682 bp, Artificial Sequence
  Synthetically generated oligonucleotide
tctagaaatggtactcgtggatgaacctaacgtcaccgaagtattgatccgggtgcacagacgcaaaattctactaaagaatccatggaccaaggaagagcaccaagttgttctactgaactatcataattctcaatacttcggtgaaataaagatcggaacacccggtaggagatttgttgtcgtgtttgatactggctcatcgaatctgtgggtaccggcagccgagggtgccggtgtggccggattgcaagcaatcagcacggctcatggattaggagcgcaggtctttggtcacgatgttaggtctgcaacgcgagaggaagttgagtcttgtggtggaaagttcatcggactcagaatgggcgaggaagctgaagttttgggtgggtatgctcgggaaatgggagatgcctaccaacgtgctcaacagagccaggttcttcttagagtatccgccccttcgccagacctggtgtcaaggattcctagggacaaggtgttgatctcctaccttttcccaagcattaaccaacaagcactagacgacctggagcagtctctcgaggccggtaagcaaggcgcagagtgcctgcttcgctctagtaagcttgctttagaggcgctcttggagggagctcgtgtcgctgctactcgtggcctcctttgacccggggtcgac

SEQ ID NO: 11 - protein, 34 aa, Artificial Sequence
  Synthetically generated peptide
VLVDEPNVTEVLIRVHRRKILLKNPWTKEEHQVV

SEQ ID NO: 12 - protein, 28 aa, Artificial Sequence
  Synthetically generated peptide
SPPSTPVSPPSTPVSPPSTPVSPPSTPV

(19) United States
(12) Patent Application Publication
(10) Pub. No.: US 2011/0078828 A1
(43) Pub. Date: Mar. 31, 2011

(54) MULTI EPITOPE VACCINE FOR POULTRY

(75) Inventor: JAMES M. MACPHERSON, SASKATCHEWAN (CA)

(73) Assignee: GUARDIAN BIOTECHNOLOGIES INC., SASKATOON (CA)

(21) Appl. No.: 12/580,972

(22) Filed: Oct. 16, 2009

Related U.S. Application Data
(63) Continuation-in-part of application No. 11/222,952, filed on Sep. 9, 2005, now abandoned.
(60) Provisional application No. 60/608,370, filed on Sep. 10, 2004.

(51) Int. Cl. A01H 5/10; C07K 14/00; C07H 21/04; C12N 15/63; C12N 1/11; A61K 39/002; C12N 15/09

(52) U.S. Cl. 800/298; 530/324; 530/350; 536/23.1; 435/320.1; 435/258.4; 530/326; 424/185.1; 435/69.3

(57) ABSTRACT

Antigenic polypeptides, capable of inducing an immune response against multiple parasites, and methods of designing such polypeptides, are provided. Also provided by the invention are polynucleotides encoding such polypeptides, as well as recombinant vectors and transformed host cells containing the said polynucleotides. Oral administration and intramuscular injection of the polypeptides provides vaccination protection against infection from Eimeria parasites that result in the poultry disease coccidiosis.

This application claims the benefit of U.S. application Ser. No. 11/222,952, filed on Sep. 9, 2005, which claims priority to provisional Application No. 60/608,370, filed Sep. 10, 2004.

### FIELD OF INVENTION

This invention relates to the field of vaccines. More particularly this invention provides vaccines for protection of poultry against parasites.

### BACKGROUND OF THE INVENTION

Coccidiosis is a serious disease of poultry that is caused by a group of obligate, intracellular protozoan parasites of the genus Eimeria. These parasites cause severe lesions within the intestines of poultry that lead to reduced weight gain, delayed maturity and often death. Worldwide, this group of parasites causes close to $1 billion (US) of economic losses yearly. Since the early 1950's, the poultry industry has used anticoccidial compounds to control this disease. However, as has occurred with bacterial infections, Eimeria parasites have rapidly developed resistance to such compounds (Greif et al., 1996, Parasitol 82: 706-714). The availability of new anticoceidal drugs has been limited by high costs of drug development, the rapid emergence of Eimeria resistance to the drugs, and to consumer demands for chemical-free agricultural products.

It is well-known that poultry become immune to the negative effects of Eimeria as a consequence of resistance developed in response to natural infection, consequently, considerable efforts have been made to develop vaccines containing live, attenuated or killed Eimeria oocysts (Vermeulen, 1998, Int. J. Parasitol. 28: 1121-1130). However, killed vaccines have failed to elicit adequate protection against Eimeria in poultry when compared to live vaccines (Danforth et al., 1993, VIth Inter. Coccidiosis Conference, pp. 49-60).

There are also major drawbacks to live vaccines which have limited their use in the poultry industry, for example, live vaccines are expensive to produce, large volumes are required for commercial flocks, they are difficult to administer in controlled doses, and there is a constant threat that live vaccines may revert to virulence (Binger et al., 1993, Mol. Biochem. Parasitol. 61: 179-188). A major problem for live vaccines for commercial use is unequal exposure to individual birds across a large flock. Factors such as uneven suspension of the parasites in the delivery liquid or pecking order can also result in unequal vaccine delivery. Vaccination with live parasites can also be problematic due to simple environmental conditions. For example, in dry environments, sporulation of the Eimeria oocysts (infective stage) may be insufficient to provide protection, while a wet environment may result in high sporulation rates creating too high of a challenge for the animal, leading to infection rather than immunization.

An alternative option for efficient and effective delivery of a vaccine to the intestine site is the production of antigenic proteins inside host cells, wherein the cell protects the antigenic agent during the digestive process. Expression inside cells of certain bacteria, yeasts or transgenic plants can provide such protection. When using transgenic plants, the plant parts are harvested, processed, and fed to the poultry as “oral vaccines” (Daniell et al., 2001, Trends Plant Sci. 6: 219-226; Giddings et al., 2000, Nature Biotechnol. 18: 1151-1155). The rigid cell walls of plants protect the antigenic proteins from digestion in the host stomach (Rigano et al., 2003, Vaccine 21, 809-811). Bacterial cellulases present in the intestines, eventually digest the plant cell wall and allow delivery of the vaccines' antigenic proteins to the intestine.

Oral vaccines produced in transgenic plants have been shown to synthesize properly folded animal and human proteins (Bouche et al., 2002, Vaccine 20:1-8). Consequently, oral administration of therapeutic proteins can produce immune responses when subsequently challenged with the pathogen (Mason et al., 1998, Vaccine 16: 1336-1343; Mason and Arntzen, 1995, C. J. Trends Biotechnol 13: 388-392).

Although the Eimeria species are closely related, immunity is strongly species-specific, i.e., each Eimeria species produces a different immune response thereby adding to the complexity of producing functionally effective Eimeria vaccines. Generally, the Eimeria species that cause the greatest economic problems are E. acervulina, E. maxima, E. tenella and E. necatrix. Therefore, a commercially effective, wide spectrum Eimeria vaccine should contain representatives of the each of these species in a single product.

Protective immunity to natural coccidiosis infection has been well documented. Controlled, daily administration of small numbers of viable oocysts for several weeks may result in complete immunity to a challenge infection of a normally virulent dose (Rose et al., 1976, Parasitology 73: 25-37; Rose et al., 1984, Parasitology 88: 199-210; U.S. Pat. No. 4,544,548; U.S. Pat. No. 4,552,759; U.S. Pat. No. 4,752,475; U.S. Pat. No. 4,863,731). The production of vaccines comprising nucleic acids encoding Eimeria proteins, and recombinant vaccines, has also been disclosed (U.S. Pat. No. 6,203,801; U.S. Pat. No. 5,661,015; U.S. Pat. No. 5,925,347; U.S. Pat. No. 5,795,741; U.S. Pat. No. 5,709,862; U.S. Pat. No. 5,635,181).

There have been numerous attempts to isolate individual long-chain antigenic proteins from different Eimeria species at different life stages for use as vaccines (Brown et al., 2000, Mol. Biochem. Parasitol. 107: 91-102; Ng et al., 2002, Exper. Parasitol. 101: 168-173; Belli et al., 2002, Inter. J. Parasitol. 32: 1727-1737; Wallach et al., 1995, Vaccine 13: 347-354). None of these documents disclose the use of a multi-species, multi epitope vaccine.

In order to achieve protection from Eimeria infections where the target organs are the intestines, the production of mucosal immunity or secretary IgA (sIgA) antibodies by the intestines is critical for successful development of immunity. A possible method to generate such antibodies is through the administration of oral vaccines. In the past this approach has been problematic due to rapid antigenic protein destruction within the host digestive system.

### SUMMARY OF THE INVENTION

This invention relates to the field of vaccines. More particularly this invention provides vaccines for protection of poultry against parasites.

It is an object of the invention to provide an improved vaccine for poultry.

The present invention relates to novel polypeptides for eliciting an immune responses to multiple species of a parasite, for example but not limited to, multiple species of the Eimeria parasite in an animal or human subject. The polypeptide can be produced by transforming a host cell with a polynucleotide encoding the polypeptide using recombinant molecular biology techniques and providing suitable conditions for expression of the transgenic polypeptide in the host cell. In a preferred embodiment of the invention the host is a plant and as the transgenic plant grows and develops, the transgenic polypeptide accumulates within certain plant parts that are subsequently harvested and processed into stable and storable forms suitable for dietary consumption.

The present invention provides a multi epitope protein (MEP) comprising two or more than two different proteins or different protein fragments, the two or more than two different proteins or different protein fragments are expressed within different life stages of a parasite or different cellular locations of the parasite, within different species of the parasite, within the parasite and one or more second parasite, or a combination thereof, wherein the MEP exhibits an antigenic response against one or more than one protein obtained from a parasite, one or more than one protein obtained from different species of the parasite, or one or more than one protein obtained from the parasite and one or more second parasite.

The present invention further provides the multi epitope protein as defined above, wherein each of the two or more than two different proteins or different protein fragments are selected from the group consisting of a surface protein, a fragment of a surface protein, a microneme protein, a fragment of a microneme protein, a refractile body, a fragment of a refractile body and a combination thereof.

The present invention also pertains to the multi epitope protein as defined above, wherein the MEP comprises an amino acid sequence selected from the group consisting of SEQ ID NO: 1, an amino acid sequence having an identity with SEQ ID NO:1 of from about 70% to about 100%, SEQ ID NO: 2, an amino acid having an identity with SEQ ID NO:2 of from about 70% to about 100%, SEQ ID NO: 3, an amino acid having an identity with SEQ ED NO:3 of from about 70% to about 100%, SEQ ID NO: 4, an amino acid having an identity with SEQ ID NO:4 of from about 70% to about 100%, SEQ ID NO: 5, and an amino acid having an identity with SEQ ID NO:5 of from about 70% to about 100%, wherein the identity is determined using BLAST, at default parameters: Program: blastp; Expect 10; filter: default; G=11; E=1; and W=3.

The present invention also provides a multi epitope protein (MEP) comprising two or more than two different proteins or different protein fragments, wherein the two or more than two different proteins are selected from the group consisting of SEQ ID N: 13-27 and wherein the two or more than two different protein fragments are selected from the group consisting of SEQ ID NOs: 11, 12, 28-41.

The present invention also provides a polynucleotide that encodes a multi epitope protein as defined herein.

According to the present invention, there is provided a polypeptide having an amino acid sequence selected from the group consisting of SEQ ID NO: 1; SEQ ID NO: 2; SEQ ID NO: 3; SEQ ID NO: 4; SEQ ID NO: 5 and fragments thereof. The polypeptide sequence of the present invention is capable of eliciting an immune response in an animal to multiple species of the Eimeria parasite.

The present invention provides a vaccine capable of eliciting an immune response in an animal to multiple species of the Eimeria parasite. The Eimeria parasite typically infects poultry causing the disease coccidiosis. It is therefore a further object of the invention to provide an improved vaccine for the treatment of coccidiosis in poultry and to provide a method of vaccinating poultry against coccidiosis.

Accordingly, there is provide by the present invention a method of immunizing poultry against coccidiosis comprising administering an effective immunizing dose of a polypeptide having an amino acid sequence selected form the group consisting of SEQ ID NO: 1; SEQ ID NO: 2; SEQ ID NO: 3; SEQ ID NO: 4; SEQ ID NO: 5 and fragments thereof. The polypeptide may be administered orally or by intramuscular injection.

The polypeptide used to immunize poultry against coccidiosis has preferably been expressed in a host cell and the host cell comprising the expressed polypeptide may be administered orally to the poultry. The host cell is preferably a plant cell and plant tissue, for example, but not limited to leaves, roots, stem, tubers, fruit, seeds, flowers, and extracts thereof, containing the expressed polypeptide may be administered to the poultry. The host cell may also be bacteria or a yeast cell.

The polypeptide of the present invention preferably elicits a mucosal immune response in the poultry.

According to one aspect of the invention, a transgenic polypeptide for use in immunizing poultry against coccidiosis may be constructed using recombinant technologies to combine amino acid sequences from recognized epitopes of different proteins selected from two or more Eimeria parasites, thereby providing a polypeptide which confers concurrent immuno-protection against two or more species of Eimeria parasites.

Therefore in accordance with the present invention, there is provided a polypeptide comprising a plurality of peptide sequences obtained from two or more species of Eimeria, preferably selected from the group consisting of E. tenella, E. acervulina, E. maxima or E. necatrix. Each of the peptide sequences are preferably shorter than about 300 amino acids.

The present invention further provides a polynucleotide that encodes a polypeptide having an amino acid sequence selected form the group consisting of SEQ ID NO: 1; SEQ ID NO: 2; SEQ ID NO: 3; SEQ ID NO: 4; SEQ ID NO: 5 and fragments thereof. The polynucleotide of the present invention preferably has a nucleotide sequence selected from the group consisting of SEQ ID NO: 6; SEQ ID NO: 7; SEQ ID NO: 8; SEQ ID NO: 9; SEQ ID NO: 10 and fragments thereof.

The present invention further provides a nucleic acid construct comprising a polynucleotide that encodes a polypeptide having an amino acid sequence selected form the group consisting of SEQ ID NO: 1; SEQ ID NO: 2; SEQ ID NO: 3; SEQ ID NO: 4; SEQ ID NO: 5 and fragments thereof, operatively linked to an expression control sequence enabling expression of the polynucleotide in a host cell.

The polynucleotide encoding the polypeptide of the present invention is preferably constructed and ligated into an appropriate plasmid vector containing selection markers, along with a promoter for regulating production of the polypeptide in a transgenic host cell wherein the promoter is inserted into the plasmid vector upstream from the polynucleotide. The plasmid vector is used to transform the host cell thereby enabling expression of the introduced polynucleotide in the host cell.

Accordingly, the present invention further provides a vector comprising a polynucleotide that encodes a polypeptide having an amino acid sequence selected form the group consisting of SEQ ID NO: 1; SEQ ID NO: 2; SEQ ID NO: 3; SEQ ID NO: 4; SEQ ID NO: 5 and fragments thereof. Preferably, the vector comprises the nucleic acid construct of the present invention.

The present invention further provides a host cell transformed with a polynucleotide that encodes a polypeptide having an amino acid sequence selected form the group consisting of SEQ ID NO: 1; SEQ ID NO: 2; SEQ ID NO: 3; SEQ ID NO: 4; SEQ ID NO: 5 and fragments thereof. The host cell is preferably a plant cell, for example, but not limited to Chlamydomonas, Brassica napus (canola) or Cucumis melo (oriental melon), the host cell may also be a bacterium for example, but not limited to E. coli or a yeast cell, for example, but not limited to S. cerevisiae.

in a further aspect of the present invention, there is provided a method of producing a polypeptide having an amino acid sequence selected form the group consisting of SEQ ID NO: 1; SEQ JD NO: 2; SEQ ID NO: 3; SEQ ID NO: 4; SEQ ID NO: 5, and a fragment thereof, comprising preparing a transgenic host cell comprising a recombinant polynucleotide encoding said polypeptide and providing suitable conditions for expression of said polypeptide by the host cell.

A further aspect of the present invention provides a transgenic plant comprising a recombinant polynucleotide encoding one or more polypeptides selected form the group consisting of SEQ ID NO: 1; SEQ ID NO: 2; SEQ ID NO: 3; SEQ ID NO: 4; SEQ ID NO: 5, and a fragment thereof. The present invention also provides a seed of the transgenic plant comprising a recombinant polynucleotide encoding one or more polypeptides selected form the group consisting of SEQ ID NO: 1; SEQ ID NO: 2; SEQ ID NO: 3; SEQ ID NO: 4; SEQ ID NO: 5, and a fragment thereof.

The transgenic plant in which the polypeptide of the present invention has been expressed and accumulated, can be harvested and processed into forms suitable for dietary consumption such as, but not limited to, powders, granules, pellets or liquids.

In accordance with a further aspect of the present invention, there is provided a bacterium comprising a recombinant polynucleotide encoding one or more polypeptides selected form the group consisting of SEQ ID NO: 1; SEQ ID NO: 2; SEQ ID NO: 3; SEQ ID NO: 4; SEQ ID NO: 5, and a fragment thereof.

A further aspect of the present invention provides a yeast cell comprising a recombinant polynucleotide encoding one or more polypeptides selected form the group consisting of SEQ ID NO: 1; SEQ ID NO: 2; SEQ ID NO: 3; SEQ ID NO: 4; SEQ ID NO: 5, and a fragment thereof.

It will be known to those skilled in the art that bacterium and yeast cells containing the polypeptide that has been expressed and accumulated in the cells can be harvested and processed into forms suitable for dietary consumption by humans or animals such as, but not limited to, powders, granules, pellets or liquids.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a functional plasmid map of plant binary vectors pHS737, pTHK-1 or pTHK-2 used for transforming a plant with the coding sequence for the MEP protein of interest, wherein the coding sequence is introduced in between the Xba I Bam HI sites in any of the vectors in accordance with the present invention. FIG. 1A shows the characteristics of vector pHS737; FIG. 1B shows the characteristics of vector pTHK-1; and FIG. 1C shows the characteristics of vector pTHK-2.

FIG. 2 shows a functional map of yeast vector YGAP. The coding sequence for the MEP protein of interest is cloned into the YGAP vector using EcoRI and XbaI sites in accordance with the present invention.

FIG. 3 shows a functional plasmid map of a vector containing the coding sequence for expression of the MEP proteins (MEP1, MEP2, MEP3, MEP4, or MEP5) in a bacterium using pGEX vector, in this example MEP1 is shown. The coding sequence is cloned into the vector using the Xba I and BamHI sites in accordance with the present invention.

FIG. 4 shows a functional plasmid map of plant plasmid vectors containing the coding sequence for expression of the MEP proteins (MEP1, MEP2, MEP3, MEP4, or MEP5) in plants using pBI121, pHS737, PTHK-1 or pTHK-2 vectors in accordance with the present invention. In this example, MEP 1 is shown.

FIG. 5 shows a western blot analysis of MEP1 protein expression in E. coli BL-21 cells. Lane I shows molecular weight markers. Lane 2 shows a pre-washing step of GST-fusion MEP proteins released from the E. coli cells. Lane 3 shows recovered proteins from the initial washing steps. Lane 4-6 show increasing purity as the GST-fusion proteins undergoes repeated rounds of washing and purification. The GST-fusion proteins were detected using anti GST antibodies as described in the Examples.

FIG. 6 shows a western blot analysis of MEP5 protein expression in yeast. Lane 1 shows molecular weight markers. Lane 7 shows the various contaminating proteins from lysed yeast cells. Lane 6 to lane 2 show purification of a GST-MEP protein fusion product. The GST-MEP fusion proteins were detected using anti-GST antibodies as described in the Examples.

FIG. 7 shows different stages in development of transformed Cucumis melo (oriental melon) plants as described in the Examples. FIG. 7A shows shoot development in LDMII media and FIG. 7B shows transformed and regenerated melon plants with emerged roots in LDMIII media.

FIG. 8 shows the sequence for MEP 1. FIG. 8A shows the amino acid sequence of MEP1 (SEQ ID NO:1). FIG. 8B shows the nucleotide sequence encoding MEP1 (SEQ ID NO:6).

FIG. 9 shows the sequence for MEP2. FIG. 9A shows the amino acid sequence of MEP2 (SEQ ID NO:2). FIG. 9B shows the nucleotide sequence encoding MEP2 (SEQ ID NO:7).

FIG. 10 shows the sequence for MEP3. FIG. 10A shows the amino acid sequence of MEP3 (SEQ ID NO:3). FIG. 10B shows the nucleotide sequence encoding MEP3 (SEQ ID NO:8).

FIG. 11 shows the sequence for MEP4. FIG. 11A shows the amino acid sequence of MEP4 (SEQ ID NO:4). FIG. 11B shows the nucleotide sequence encoding MEP4 (SEQ ID NO:9).

FIG. 12 shows the sequence for MEP5. FIG. 12A shows the amino acid sequence of MEP5 (SEQ ID NO:5). FIG. 12B shows the nucleotide sequence encoding MEP5 (SEQ ID NO:10).

FIGS. 13A-O show the amino acid sequences of various exemplary proteins from which MEPs may be constructed.

FIG. 14 is a graph showing average bird weights on day 6, post infection.

FIG. 15 is a graph showing average bird weight gains on day 10, post infection.

FIG. 16 is a graph showing average bird weight gains on day 18, post infection.

This invention relates to the field of vaccines. More particularly this invention provides vaccines for protection of poultry against parasites.

### DETAILED DESCRIPTION

The following description is of the preferred embodiments.

The present invention provides methods for identifying epitopes from various immunogenic proteins. By “epitope” is meant a peptide sequence of about 6 to about 12 amino acids, or any value therebetween, such as 7, 8, 9, 10 or 11. An epitope may be a continuous stretch of amino acids in the primary structure of a polypeptide, or may be a series of amino acids that are separated in the primary structure of a polypeptide, but spatially proximate in the tertiary structure.

An epitope may be identified by examining a polypeptide sequence for stretches that are hydrophilic, located on the protein surface, and are flexible (Trends Biochem (1986) 11:36-39; Proc. Natl. Acad. Sci. USA (1981) 78: 3824-3828). In general, biologically active proteins have hydrophilic regions located on the surface., while hydrophobic regions are buried in the interior of the protein tertiary structure. In addition, the secondary structure of a protein is also relevant with respect to identification of epitopes—proteins consist of secondary structures such as alpha-helix, beta-sheets and beta-turn regions. While each of these may have antigenic properties, beta-turns are often on the outside of the protein and arc highly flexible (J. Mol. Biol. (1978) 120: 97-120). Epitopes may be predicted using software programs that use these characteristics to predict epitopes with a high degree of accuracy, such as those referenced in: Immunome Res. (2008) 4:1; Bioinformatics (2006) 22: 1088-1095; J. Computational Biol. (2007) 14: 736-746; Proc. Computational Systems Bioinformatics Conference (2003) pp 17-26; Eur. J. Immunol. (2006) 35: 2295-2303 or Mol. Innumol. (2006) 43: 2037-2044. Such software programs for predicting the above protein characteristics such as hydrophilic regions and secondary (folding) structures aid in the selection of potentially exposed regions of the protein that serve as epitopes. Further, computer algorithms are available that predict, using the hydrophobic natures of the proteins, the most likely interior and exterior regions of a protein and aid in the 3D modeling of protein structures (J. Mol. Bio. (1982) 157: 105-132; Protein Sci. (2006) 15: 2558-2567). These surface regions or regions of high accessibility are often accompanied with beta-turns that have been found to be antigenic (Peptide Res. (1991) 4: 347-354). The combination of two or more predictive algorithms lead to epitope prediction success rates as high as 86% (Peptide Res.(1991) 4: 355-363; Proc. Natl. Acad. Sci. USA (1988) 85: 5409-5413).

Additional strategies for determining suitable epitopes may be found in the art, as referenced in for example M J Blythe, I A Doytchinova, and D R Flower. JenPep: a database of quantitative functional peptide data for immunology. Bionformatics 2002 18 434-439; Doytchinova, I. A and Flower, D. R. Towards the quantitative prediction of T-cell epitopes: CoMFA and CoMSIA studies of peptides with affinity to class 1 MHC molecule HLA-A*0201. J. Med. Chem. 2001, 44, 3572-3581; Irini A. Doytchinova, Martin J. Blythe and Darren R. Flower An Additive Method for the Prediction of Protein-Peptide Binding Affinity. Application to the MHC Class I Molecule HLA-A*0201 J. Proteose Research 2002, 1, 263-272; or Doytchinova, I. A and Flower, D. R. Physicochemical Explanation of Peptide Binding to HLA-A*0201 Major Histocompatibility Complex. A Three-Dimensional Quantitative Structure—Activity Relationship Study Proteins. 2002 Aug. 15; 48(3):505-18.

Once potential epitopes are identified, the length of the peptide is determined. While an epitope can be as short as about 6 to about 12 amino acids, or any value therebetween, the immune response to a sequence of this length may not be sufficiently strong. Accordingly, in one aspect of the invention, selected epitope-containing sequences from different proteins are presented in the context of a longer polypeptide that is large enough to stimulate the immune system. Such chimeric polypeptides are designated as “multi-epitope proteins” or “MEPs” according to the present invention. An MEP according to the invention may range from about 15 to about 1,000 amino acids in length or any length therebetween, such as about 20, 22, 25, 30, 35, 40, 45, 50, 60, 80, 100, 150, 200, 250, 300, 350, 400, 450, 500, 550, 600, 650, 700, 750, 800, 850, 900, 950 amino acids.

In general, the MEPs are designed to produce antigens of between 20-50 Kda to make the antigens easily presentable to the immune system. It is to be understood that the order of fragments in the recombinant protein is not significant, and that the fragments may be combined in any order.

Eimeria parasites at the sporozoite stage of the life cycle were selected as an exemplary target of the epitope selection and MEP generation process. A list of antigenic proteins identified in different Eimeria species was prepared, and the antigens were classified according to their location in the parasite as well as the developmental stage at which they were produced. Fifteen sequences were classified in four groups characterized as antigens expressed at the surface, antigens produced by micronemes, antigens produced by the gametocytes and antigen produced by the retractile body, as follows:

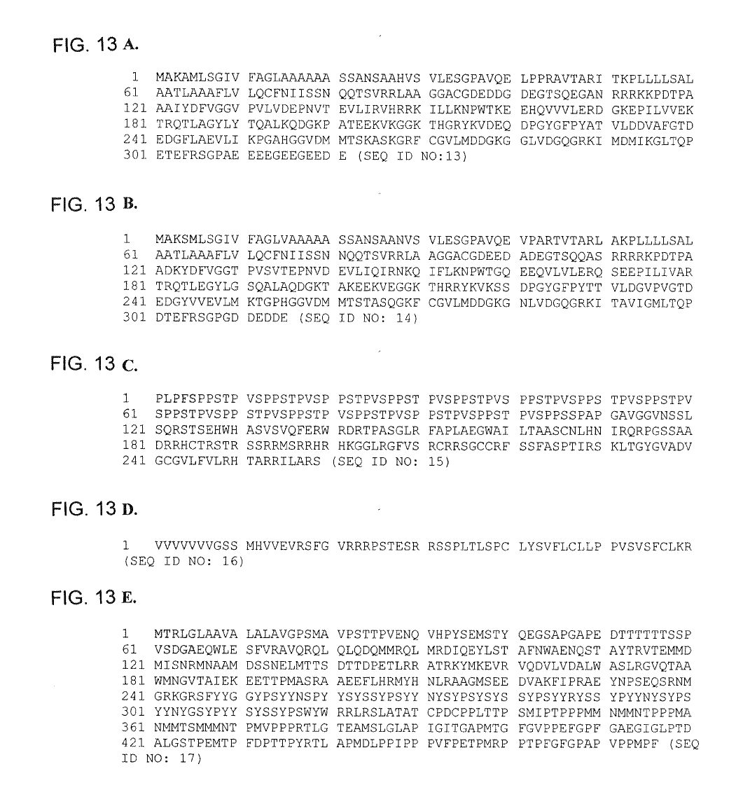

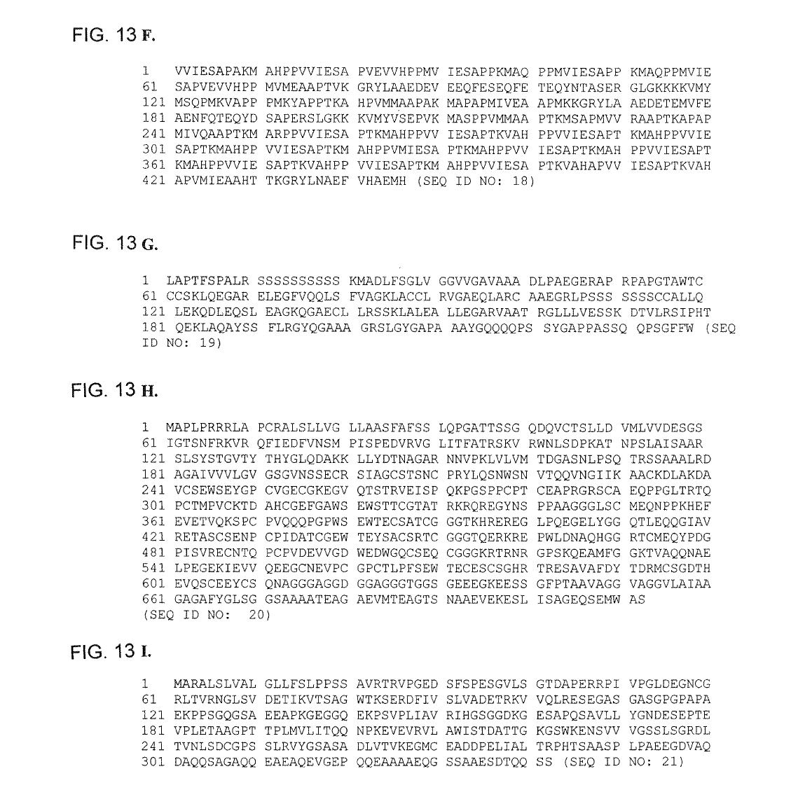

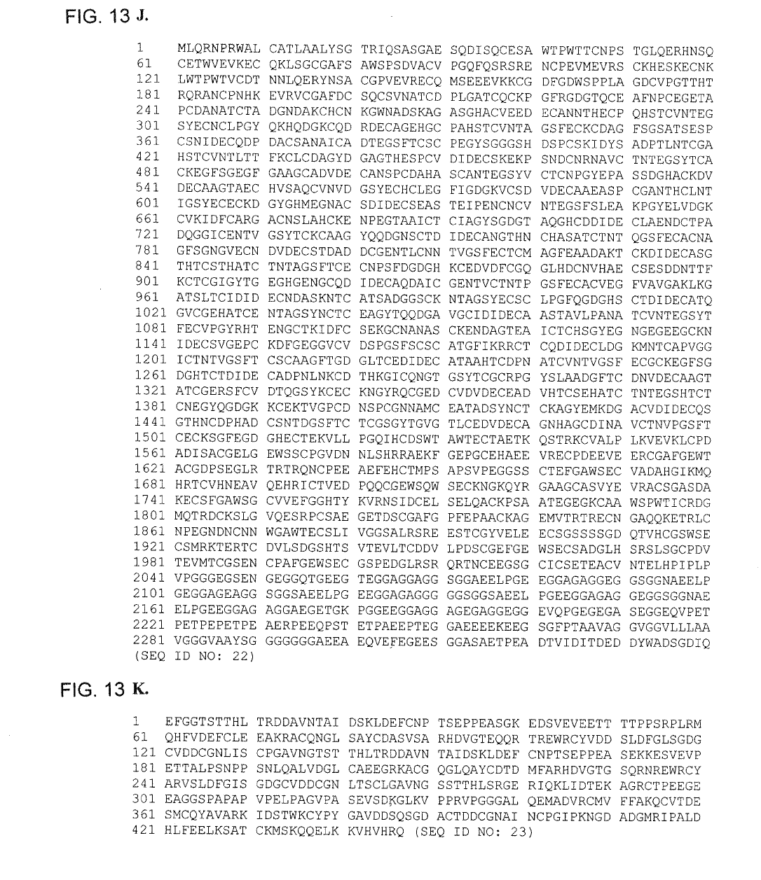

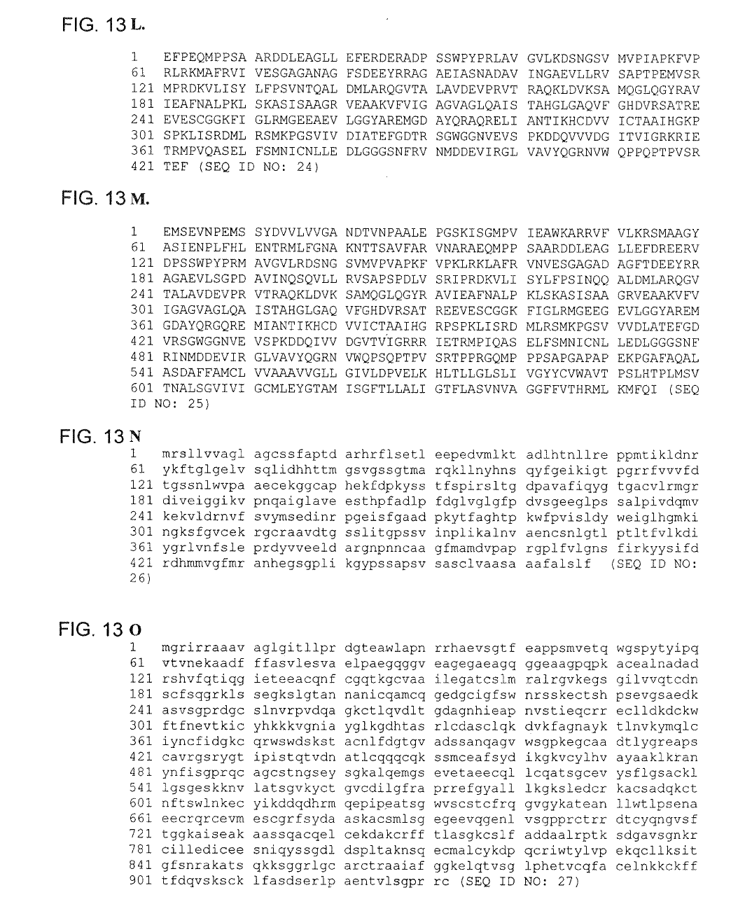

NPmz19 of Eimeria necatrix (a surface protein; Tajima et al., 2003, Avian Dis. 47: 309-318; FIG. 13A, Accession No. BAB85126, SEQ ID NO: 13);

Mzp5-7 of Eimeria tenella (merozoite surface protein, a surface protein; Binger et al., 1993, Mol. Biochem. Parasitol 61: 179-187; FIG. 13B, Accession No. AAA 16457, SEQ ID NO: 14);

Eamzp35 of Eimeria acervulina (merozoite surface protein, a surface protein; Jenkins, 1988, Nucl. Acids Res. 16: 9863; FIG. 13C, Accession No. CAA30977, SEQ ID NO: 15);

Easz22 of Eimeria acervulina (sporozoite surface antigen, a surface protein; Jenkins et al, 1989, Mol. Biochem. Parasitol. 32: 153-161; FIG. 13D, Accession No. AAA29078, SEQ ID NO: 16);

56 KDa of Eimeria maxima (oocysts with protein, a surface protein; Belli et al, 2002, Int. J. Parasitol. 32: 1727-1737; FIG. 13E, Accession No. AAN05087, SEQ ID NO: 17);

230 KDa of Eimeria maxima (macrogamete-specific protein, a surface protein; 1992, Mol. Biochem. Parasitol. 51: 251-262; FIG. 13F, Accession N AAB02122, SEQ ID NO: 18);

S07 of Eimeria acervulina (sporozoite surface antigen, a surface protein; Liberator et al, 1989, Nucl. Acids Res. 17: 7104; FIG. 13G, Accession No. CAA33905, SEQ ID NO: 19);

Etmic1 of Eimeria tenella (a microneme protein; Tomley et al, 1991, Mal. Biochem. Parasitol. 49: 277-288; FIG. 13H, Accession No. AAD03350, SEQ ID NO: 20);

Etmic2 of Eimeria tenella (a microneme protein; Tomley et al, 1996, Mol. Biochem. Parasitol. 79: 195-206; FIG. 13I, Accession No. CAA96437, SEQ ID NO: 21);

Etmic4 of Eimeria tenella (a microneme protein; Tomley et al, 2001, Int. J. Parasitol. 31:1303-1310; FIG. 13J, Accession No. CAC34726, SEQ ID NO: 22);

Etmic5 from Eimeria tenella (microneme protein), Brown et al., Mol. Biochem. Parasitol. (2000) 107: 91-102 (FIG. 13O, Accession No. CAB52368, SEQ ID NO: 27)

Em100 of Eimeria acervulina (a microneme protein; Pasamontes, et al, 1993, Mol. Biochem. Parasitol. 57: 171-174; FIG. 13K, Accession No. AAA29076 SEQ ID NO: 23);

p43 of Eimeria (a refractile body; Laurent et al, 1993, Mol. Biochem. Parasitol. 62303-312; FIG. 13N, Accession No 396454. SEQ ID NO: 26);

Ea1A of Eimeria acervulina (transhydrogenase; a refractile body protein; Vermeulen et al, 1993, FEMS Microbiol. Lett. 110: 223-229; FIG. 13L, Accession No. AAA61928, SEQ ID NO: 24);

Eta1A of Eimeria tenella (transhydrogenase, a refractile body protein; Vermeulen et al, 1992; FIG. 13C, Accession No. AAA29081, SEQ ID NO: 25).

For each group, the proteins were aligned using online sequence software (Clustal) to provide an overall assessment of relatively conserved regions that function as core or structural regions. In addition, hydrophilic and hydrophobic regions were identified using the algorithm ANTIGENIC which is based upon the prediction methods described by Hopp and Woods (Proc. Natl. Acad. Sci. USA (1981) 78: 3824-3828). Finally, the algorithm GARNIER as originally described by Garnier et al (J. Mol. Biol. (1978) 120: 97-120), which predicts secondary structure such as alpha-helices, beta-sheets or beta-turns, was combined with ANTIGENIC to locate potential antigenic regions that included beta-turns and were hydrophilic. These antigenic regions or epitopes were combined to construct MEPs.

Accordingly, in one aspect, the present invention provides MEPs for eliciting an immune response to one or multiple species of a parasite in an animal, for example but not limited to the Eimeria, Toxoplasma, Cryptospordium, Sarcocystis or Plasmodium parasite. These MEPs are comprised of protein domains, fragments of protein domains, or functionally equivalent proteins or protein fragments of two or more proteins obtained from one or more parasites. By a functionally equivalent protein, it is meant that the protein or fragment of the protein comprises an amino acid sequence that is modified by deletions, insertions or substitutions without essentially changing the immunological properties of the protein or polypeptide. It is to be understood that MEPs include nucleic acid molecules encoding the polypeptide sequences of the MEPs.

The MEP of the present invention may be prepared using recombinant technologies known in the art to combine amino acid sequences from epitopes of different proteins selected from one or more than one parasite, for example but not limited to two or more Eimeria, Toxoplasma, Cryptospordium, Sarcocystis or Plasmodium parasites, into a chimeric protein. In this example the MEP provides a polypeptide that confers concurrent immuno-protection against two or more species of a parasite.

Proteins or peptides may also be identified from different species of a parasite, for example, Eimeria. In this example, which is not to be considered limiting, the species may include E. tenella, E. acervulina, E. maxima or E. necatrix. The proteins or peptides may be identified at different life stages, within different cellular locations or both, from different species of Eimeria. For example, a protein or a fragment of a protein may be obtained from, but are not limited to, a surface protein or a fragment thereof, a microneme protein or a fragment thereof, a retractile body or a fragment thereof, and each of these proteins or a fragment thereof may be used as a subunit within the MEP. Non-limiting examples of proteins that may be used as a source of peptide subunits for the preparation of an MEP include:

NPmz19 of Eimeria necatrix (a surface protein; Tajima et al., 2003, Avian Dis. 47: 309-318; FIG. 13A, Accession No. BAB85126, SEQ ID NO: 13);

Mzp5-7 of Eimeria tenella, (a surface protein; Binger et al., 1993, Mol. Biochem. Parasitol 61: 179-187; FIG. 13B, Accession No. AAA16457, SEQ ID NO: 14);

Eamzp35 of Eimeria acervulina (a surface protein; Jenkins, 1988, Nucl. Acids Res. 1.6: 9863; FIG. 13C, Accession. No. CAA30977, SEQ ID NO: 15);

Easz22 of Eimeria acervulina (a surface protein; Jenkins et al, 1989, Mol. Biochem. Parasitol. 32: 151-161; FIG. 13D, Accession No. AAA29078, SEQ ID NO: 16);

56 KDa of Eimeria maxima (a surface protein; Belli et al, 2002, Int. J. Parasitol. 32: 1727-1737; FIG. 13E, Accession No. AAN05087, SEQ ID NO: 17);

230 KDa of Eimeria maxima (a surface protein; 1992, Mol. Biochem. Parasitol. 51: 251-262; FIG. 13F, Accession No. AAB02122, SEQ ID NO: 18);

S07 of Eimeria acervulina (a surface protein; Liberator et al, 1989, Nucl. Acids Res. 17: 7104; FIG. 13G, Accession No. CAA33905, SEQ ID NO: 19);

Etmic1 of Eimeria tenella (a microneme protein; Tomley et al, 1991, Mol. Biochem. Parasitol. 49: 277-288; FIG. 13H, Accession No. AAD03350, SEQ ID NO: 20);

Etmic2 of Eimeria tenella (a microneme protein; Tomley et al, 1996, Mol. Biochem. Parasitol. 79: 195-206; FIG. 13I, Accession No. CAA96437, SEQ ID NO: 21);

Etmic4 of Eimeria tenella (a microneme protein; Tomley et al, 2001, Int. J. Parasitol. 31:1303-1310; FIG. 13J, Accession No. CAC34726, SEQ ID NO: 22);

Etmic5 from Eimeria tenella (microneme protein), Brown et al., Mol. Biochem. Parasitol. (2000) 107: 91-102 (FIG. 13O, Accession No. CAB52368, SEQ ID NO: 27)

Em100 of Eimeria acervulina (a microneme protein; Pasamontes, et al, 1993, Mol. Biochem. Parasitol. 57: 171-174; FIG. 13K, Accession. No. AAA29076, SEQ ID NO: 23);

p43 of Eimeria (a refractile body; Laurent et al, 1993, Mol. Biochem. Parasitol. 62: 303-312; FIG. 13N, Accession No. 396454 SEQ ID NO: 26);

Ea1 A of Eimeria acervulina (a retractile body; Vermeulen et al, 1993, FEMS Microbiol. Lett. 110: 223-229; FIG. 13L, Accession No. AAA61928, SEQ ID NO: 24);

Eta1A of Eimeria tenella (a refractile body; Vermeulen et al, 1992; FIG. 13C, Accession No. AAA29081, SEQ ID NO: 25).

As one of skill in the art would appreciate, different combinations of proteins or peptide subunits may be used to produce an MEP. Therefore, the selection of proteins or polypeptides, and the hosts from which these proteins or polypeptides are selected are not to be considered limiting in any manner. Furthermore, it is to be understood that other proteins or polypeptides from E. tenella, E. acervulina, E. maxima or E. necatrix, other proteins or polypeptides from other species of Eimeria, or other parasites, for example Toxoplasma, Cryptospordium, Sarcocystis or Plasmodium, may also be used to generate chimeric proteins (MEP's) that are efficacious in vaccinating an animal subject using the methods as described herein.

The multiple epitope protein (MEP) of the present invention may comprise the entire amino acid sequence of the native antigen of the parasite from which it is derived. However, in certain preferred embodiments of the invention, the MEP may represent only a portion of the native molecule's sequence. In either case, the antigen may be fused to another peptide, polypeptide or protein to form a chimeric protein or MEP using techniques well known to those of skilled in the art. Formation of an MEP can be accomplished by combining epitopes from several different antigenic proteins. In a preferred embodiment, these antigenic proteins can be from different parasites as well as from different life stages. Thus, antigens may derive from Eimeria, Toxoplasma, Cryptospordium, Sarcocystis or Plasmodium parasites.

Each of the peptide subunits that comprise the MEP of the present invention have a length sufficient to elicit a response in a target animal. The peptide subunits may comprise from about 15 amino acids to the full-length protein. Preferably, the MEP is of about 15 to about 1,000 amino acids in length or any length therebetween, from about 20 to about 500 amino acids in length, or any length there between, from about 22 to about 300 amino acids in length, or any length there between, from about 25 to about 200 amino acids in length, or any length there between. In non-limiting examples provided herein, the amino acid sequence of SEQ ID NO: 1; SEQ ID NO: 2; SEQ ID NO: 3; SEQ ID NO: 4; and SEQ ID NO: 5, are between 129 and 349 amino acids.

Preferably, two or more than two proteins or protein fragments (peptide subunits) are fused together to make an MEP. Furthermore it is preferred that the proteins, or protein fragments are obtained from different proteins, or fragments of different proteins, so that the MEP is a chimeric protein. By different proteins, or fragments of different proteins, it is meant that the proteins or protein fragments are expressed within either the same parasite but from different life stages, different cellular locations, and may comprise for example but not limited to, a surface protein or a fragment thereof, a microneme protein or a fragment thereof, a refractile body or a fragment thereof. The protein or protein fragments that comprise the MEP may also be expressed within different species of a parasite, or expressed within different parasites, and comprise proteins or protein fragments from one or more than one life stage, one or more than one cellular location, for example but not limited to, a surface protein or a fragment thereof, a microneme protein or a fragment thereof, a refractile body or a fragment thereof. In this manner at least two of the proteins or protein fragments within an MEP are different with respect to each other and the MEP is chimeric. The MEP may comprise from 2 to 20 subunits, or any number therebetween, from 3 to 10 subunits or any number therebetween, or for example 2, 3, 4, 5, 6, 7, 8, 9, 10, 11, 12, 13, 14, 15, 16, 17, 18, 19 or 20 subunits, or any number therebetween.

The present invention also includes an MEP comprising a plurality of subunit epitopes obtained from two or more species of Eimeria, preferably selected from the group consisting of E. tenella, E. acervulina, E. maxima or E. necatrix. Preferably the MEP comprises a fragment of NPmz19, for example but not limited to a fragment of the surface protein from E. necatrix. Examples of a fragment of the surface protein from E. necatrix that may be used within an MEP include, but are not limited to:

| | |
|---|---|
| VLVDEPNVTEVLIRVHRRKILLKNPWTKEEHQVV, | (SEQ ID NO: 11) |
| or | |
| SPPSTPVSPPSTPVSPPSTPVSPPSTPV. | (SEQ ID NO: 12) |

Therefore, the present invention provides an MEP comprising two or more subunits, where one of the subunits is an NPmz19 surface protein or a fragment of an NPmz19 surface protein. Multiple epitope proteins (MEP's) of the present invention also include, but are not limited to, a protein having:

the primary structural conformation of amino acids as shown in any of SEQ ID NO: 1; SEQ ID NO: 2; SEQ ID NO: 3; SEQ ID NO: 4; and SEQ ID NO: 5;

a polypeptide having at least 70% identity to the polypeptide having the primary structural conformation of amino acids as shown in any of SEQ ID NO: 1; SEQ ID NO: 2; SEQ ID NO: 3; SEQ ID NO: 4; and SEQ ID NO: 5;

a polypeptide having at least 80% identity to the polypeptide having the primary structural conformation of amino acids as shown in any of SEQ ID NO: 1; SEQ ID NO: 2; SEQ ID NO: 3; SEQ ID NO: 4; and SEQ ID NO: 5;

a polypeptide having at least 90% identity to the polypeptide having the primary structural conformation of amino acids as shown in any of SEQ ID NO: 1; SEQ ID NO: 2; SEQ ID NO: 3; SEQ ID NO: 4; and SEQ ID NO: 5; or a polypeptide having least 95% identity to the polypeptide having the primary structural conformation of amino acids as shown in any of SEQ ID NO: 1; SEQ ID NO: 2; SEQ ID NO: 3; SEQ ID NO: 4; and SEQ ID NO: 5.

provided that the MEP exhibits an antigenic response, for example, the chimeric polypeptide may exhibit the property of priming the mucosal immune system, stimulating the humoral immune response, or both. Preferably, the MEP exhibits an antigenic response against two or more species of parasite.

As used herein, the term “identity”, as known in the art, is the relationship between two or more polypeptide sequences (or two or more polynucleotide sequences), or nucleic acid sequences encoding the polypeptide sequences, as determined by comparing the sequences. In the art, identity also means the degree of sequence relatedness between polypeptide (or polynucleotide) sequences, as determined by the match between strings of such sequences. According, “identity” as used herein is meant to include a amino acid or nucleic acid sequence that differs from a reference sequence only by one or more conservative substitutions, as discussed herein, or by one or more non-conservative substitutions, deletions, or insertions located at positions of the sequence that do not destroy the biological function of the amino acid or nucleic acid molecule. Such a amino acid or nucleic acid sequence can be any integer from 10% to 99%, or more generally at least 10%, 20%, 30%, 40%, 50, 55% or 60%, or at least 65%, 75%, 80%, 85%, 90%, or 95%, or as much as 96%, 97%, 98%, or 99% identical when optimally aligned at the amino acid or nucleotide level to the sequence used for comparison. Identity can be readily calculated. For example, such determinations may be made using polypeptide alignment algorithms for example, but not limited to BLAST (GenBank URL: ncbi.nlm.nih.gov/cgi-bin/BLAST/), using default parameters (Program: blastp; Expect 10; filter: default; G=11, cost to open a gap; E=1, cost to extend a gap; and W=3 word size, default is 3).

The MEP's described in this invention can be made using a variety of cell production systems, for example plants such as but not limited to canola, cucumber, melon, potato, soybean, alfalfa, barley, wheat, grasses, plant cells, green algae such as but not limited to Chlamydomonas reinhardtii, mammalian cells, insect cells, yeast, for example Saccharomyces cerevisiae, and bacteria such as but not limited to Escherichia coli or Bacillus subtilis. Plants and plant cells expressing MEPs can be readily grown and processed, and yeast and bacterial cells expressing MEPs can be fermented and added to animal or poultry feed and function as an aid in controlling parasite, for example Eimeria, infections.

If desired, the codons of nucleotide sequences encoding the MEP may be optimized for the host organism expressing the construct. By “codon optimization” it is meant the selection of appropriate DNA nucleotides for the synthesis of oligonucleotides of a sequence encoding an MEP of the present invention using codons that are typically utilized within the target host. In order to maximize expression levels and transgene protein production of an MEP, the nucleic acid sequence of an MEP is examined and the coding region modified to optimize for expression of the gene in the target host. In order to maximize expression levels and transgene protein production, the gene may be examined at the DNA level and then the coding region optimized for expression in the target host. The standard deviation of codon usage, a measure of codon usage bias, can be calculated by first finding the squared proportional deviation of usage of each codon of the native gene relative to that of genes highly expressed in the target host, followed by a calculation of the average squared deviation. The formula used may be:

$$SDCU = {*}_{n = 1}^{N} [ ( Xn - Yn ) / Yn ] 2 / N$$

Where Xn refers to the frequency of usage of codon ‘n’ in highly expressed genes in the target host, where Yn to the frequency of usage of codon ‘n’ in the gene encoding the MEP, and N refers to the total number of codons in the MEP. A table of codon usage from highly expressed genes of the desired host may be found using codon usage tables. For example if the target host is a plant, then, a table of codon usage from highly expressed genes of dicotyledonous plants may be compiled for example using the data of Murray et al. (Nuc Acids Res. 17:477-498; 1989). However, other codon usage tables may be used.

Transformation of a suitable host cell are well known in the art and (for example, Maniatis et al, 1982, Molecular Cloning: A Laboratory Manual, Cold Spring Harbor, or Ausubel, et al, (eds), 1989, Current Protocols in Molecular Biology, Vol. 1, Green Publishing Associates, Inc., and John Wiley & Sons, Inc., New York) can be accomplished by a variety of means well described in the art such as Agrobacterium mediated transformation, microprojectile bombardment, transfection or electroporation. Regardless of the cell transforming method, once a modified host cell is generated it can be cultured and the MEP can be expressed and isolated. Alternatively, the host cell expressing the MEP can be used directly in this invention as oral inoculants.

Accordingly, the present invention further provides a host cell transformed with a polynucleotide that encodes an MEP comprising two or more than two different proteins, fragments of different proteins, or a combination thereof, obtained from a surface protein, a microneme protein, a refractile body, or a combination thereof, from one or more than one parasite for example one or more than one Eimeria species. Preferably, the MEP comprises an amino acid sequence selected form the group consisting of SEQ ID NO: 1; SEQ ID NO: 2; SEQ ID NO: 3; SEQ ID NO: 4; SEQ ID NO: 5 and antigenic fragments thereof, wherein the MEP exhibits an antigenic response against proteins from two or more than two different cellular locations, two or more than two life stages, two or more than two species of parasite, or a combination thereof.

There is further provided by the present invention a method of producing an immunogenic MEP comprising two or more than two different proteins, fragments of different proteins, or a combination thereof, obtained from proteins from different life stages, different cellular locations, or both, for example, a surface protein, a microneme protein, a refractile body, or a combination thereof, from one or more than one parasite for example. The method involves preparing a transgenic host cell comprising a recombinant polynucleotide encoding the MEP and providing suitable conditions for expression of the polypeptide by the host cell. Preferably, the immunogenic MEP comprises an amino acid sequence selected form the group consisting of SEQ ID NO: 1; SEQ ID NO: 2; SEQ ID NO: 3; SEQ ID NO: 4; SEQ ID NO: 5, and antigenic fragments thereof, wherein the MEP exhibits an antigenic response against proteins from two or more than two different cellular locations, two or more than two life stages, two or more than two species of parasite, or a combination thereof.

The MEP of the present invention is preferably a mucosal antigen. For the purposes of the invention, a mucosal antigen is an antigen that has the ability to specifically prime the mucosal immune system. More specifically, mucosal antigens are those that prime the mucosal immune system, stimulate the humoral immune response in a dose-dependent manner, without inducing systemic tolerance and without the need for excessive doses of antigen, or both prime the mucosal immune system and stimulate the humoral immune response.

Systemic tolerance is defined herein as a phenomenon occurring with certain antigens that are repeatedly fed to animals and result in a specifically diminished subsequent anti-antigen response. The MEP of the present invention, when used to induce a mucosal response, may also induce a systemic tolerance. The same MEP when introduced parenterally will typically retain its antigenicity without developing tolerance.

A mucosal response to the MEP of the present invention is understood to include any response generated when the polypeptide interacts with a mucosal membrane. Typically, such membranes will be contacted with the MEP of the present invention through feeding of the MEP orally to a subject animal, for example, but not limited to, poultry. This route of introduction provides access of the MEP to the small intestine M cells that overlie the Peyer's Patches and other lymphoid clusters of the gut-associated lymphoid tissue (GALT). However, any mucosal membrane accessible for contact with the polypeptide of the invention is specifically included within the definition of such membranes, for example, but not limited to, mucosal membranes of the air passages accessible by inhaling, mucosal membranes of the terminal portions of the large intestine accessible by suppository and the like. Thus, the MEP of the present invention may be used to induce both mucosal as well as humoral responses.

Methods of administering the MEP of the invention are also provided. Such methods comprise administering a therapeutic amount of the MEP to an animal, for example poultry. In more specific embodiments, these methods entail introduction of the MEP either parenterally or non-parenterally into the animal. Where a non-parenteral introduction mode is selected, the MEP may be orally administered by any means. Whichever mode of administration of the MEP is selected, it will be understood by those skilled in the art of vaccination that the selected mode should achieve immunization at the lowest dose possible in a dose-dependent manner and by so doing elicit serum and/or secretary antibodies against the MEP with minimal induction of systemic tolerance. Where a mucosal route of immunization is selected, care should be taken to introduce the MEP into the intestinal lumen of the target species.

Where the MEP of the present invention is subjected to adequate levels of purification as further described herein, these MEPs may also be introduced in an animal parenterally, such as by muscular injection. Similarly, while preferred embodiments of the invention include feeding of relatively unpurified MEP preparations, for example, but not limited to portions of edible plants, purees of such portions of plants, and the like, the introduction of the MEP to stimulate the mucosal response may occur through first subjecting the host cell source of the MEP to various purification procedures.

in a preferred aspect of the invention the transgenic host is a plant and such plant-derived vaccines may take various forms including purified and partially purified plant derived immunogenic polypeptides of the present invention as well as whole plant, whole plant parts such as fruits, leaves, stems, tubers, seeds as well as crude extracts of the plant or plant parts. In one embodiment, the oral vaccine of the present invention is produced in edible transgenic plants and then administered through the consumption of a part of those edible plants. A polynucleotide encoding the expression of the MEP of the present invention may be isolated and ligated into a plasmid vector containing selection markers. A promoter, which regulates the production of the MEP in the transgenic plant, may be included in the same plasmid vector upstream from the coding sequence for the MEP to ensure that the MEP is expressed in desired tissues of the plant. Preferably, the MEP is expressed in a portion of the plant that is edible by animals, such as, but not limited to poultry. For some uses, it is preferred that the edible food be a juice from the transgenic plant, which can be taken orally.

In general, the preferred state of the composition of matter which is used to induce an immune response, for example, but not limited to whole bacterium, yeast, plant, plant part, crude plant extract, partially purified MEP, or extensively purified. MEP, will depend upon the ability of the MEP to elicit a mucosal response, the dosage level of the MEP required to elicit a mucosal response, and the need to overcome interference of mucosal immunity by other substances in the chosen composition of matter for example sugars, pyrogens, toxins, chlorophyll and the like.

In another embodiment, the vaccines (oral and otherwise) are provided by deriving the MEP of the present invention from the transgenic hosts in at least a semi-purified form prior to inclusion into a vaccine. The present invention produces vaccines inexpensively. Further, vaccines from transgenic hosts can be produced in large volumes and can be administered orally, thereby reducing cost. The production of an oral vaccine in edible transgenic plants and other transgenic hosts, such as bacterium and yeast, may avoid much of the time and expense required for regulatory approval compared with purified vaccine. A principal advantage of the present invention is production of inexpensive oral vaccines, which can be used in lesser-developed countries that cannot afford or provide refrigeration required for conventional vaccines.

Thus, the present invention provides a recombinant parasite MEP, for example an Eimeria parasite MEP, expressed in a host cell. The MEP is known to elicit an antigenic response in an animal, such as, but not limited to poultry. Preferably, the MEP of the invention will be one that is known to function as an antigen when expressed in standard pharmaceutical expression systems such as yeasts or bacteria or where the polypeptide is recovered from mammalian or avian sera and shown to be antigenic. More preferably still, the MEP of the present invention will be a polypeptide known to be antigenic when used to induce an immune response through an oral mode of introduction. In the most preferred embodiment, the MEP of the present invention, known to be antigenic in its native state, will be a polypeptide, which upon expression in the host cell of the invention, retains at least some portion of the antigenicity it possesses in the native state.

Coccidiosis is a serious disease of poultry that is caused by a group of obligate, intracellular protozoan parasites of the genus Eimeria. Accordingly, the present invention provides a method of immunizing poultry against coccidiosis comprising administering an effective immunizing dose of an MEP comprising two or more than two different proteins, fragments of different proteins, or a combination thereof, obtained from a surface protein, a microneme protein, a refractile body, or a combination thereof, from one or more than one Eimeria species. Preferably the MEP used to immunize poultry against coccidiosis comprises an amino acid sequence selected form the group consisting of SEQ ID NO: 1; SEQ ID NO: 2; SEQ ID NO: 3; SEQ ID NO: 4; SEQ ID NO: 5 and antigenic fragments thereof; wherein the MEP exhibits an antigenic response against proteins from different cellular locations, life stages, species of parasite, or a combination thereof; for example, from two or more than two different cellular locations, two or more than two life stages, two or more than two species of parasite, or a combination thereof.

The MEP used to immunize poultry against coccidiosis has preferably been expressed in a host cell and the host cell, an extract of the host cell, or a purified form of the MEP, comprising the expressed polypeptide, may be administered orally to the poultry. The transgenic host may be a bacterium, yeast or another fungal species, an algae, or a multicellular plant as described above.

The present invention further provides a polynucleotide that encodes an MEP that encodes an immunogenic MEP comprising two or more than two different proteins, fragments of different proteins, or a combination thereof, that are expressed in a parasite at different life stages, different cellular compartments, or a combination thereof; for example from a surface protein, a microneme protein, a refractile body, or a combination thereof, from one or more than one parasite. For example, the nucleic acid may encode epitopes of different proteins selected from two or more parasites, for example but not limited to two or more Eimeria, Toxoplasma, Cryptospordium, Sarcocystis or Plasmodium parasites, so that when expressed, a chimeric protein (MEP) is produced that confers concurrent immuno-protection against, for example but not limited to, different life stages of one or more parasites, or against two or more species of a parasite. The nucleic acid may encode one or more than one of a protein or a fragment of a protein including but not limited to, a surface protein or a fragment thereof, a microneme protein or a fragment thereof; a refractile body or a fragment thereof. Preferably, the polynucleotide encodes an MEP that exhibits an antigenic response against proteins from different cellular locations, life stages, species of parasite, or a combination thereof, for example, from two or more than two different cellular locations, two or more than two life stages, two or more than two species of parasite, or a combination thereof.

The present invention also provides a polynucleotide that encodes an MEP having an amino acid sequence selected form the group consisting of SEQ ID NO's: 1-5 and fragments thereof. As one of skill in the art would recognize, a range of nucleic acid sequences that account for degeneracy in the genetic code may encode each of the MEP sequences defined in SEQ ID NO's: 1-5. Therefore degenerate sequences are also included within the nucleic acid sequences that may encode SEQ ID NO's:1-5.

The polynucleotide preferably has a nucleotide sequence selected from the group consisting of SEQ ID NO's: 6-10, fragments thereof, and sequences that exhibit 70% or greater, for example from about 75% to about 100%, or any amount therebetween, similarity with the nucleic acid sequences defined in SEQ ID NO's: 6-10. Such similarity determinations may be made using oligonucleotide alignment algorithms for example, but not limited to a BLAST (GenBank URL: www.ncbi.nlm.nih.gov/cgi-bin/BLAST/, using default parameters: Program: blastp; Database: nr; Expect 10; filter: default; Alignment: pairwise; Query genetic Codes: Standard (1)) or FASTA, again using default parameters.

An alternative indication that two nucleic acid sequences are substantially identical is that the two sequences hybridize to each other under moderately stringent, or preferably stringent, conditions. Hybridization to filter-bound sequences under moderately stringent conditions may, for example, be performed in 0.5 M NaHPO₄, 7% sodium dodecyl sulfate (SDS), 1 mM EDTA at 65° C. from about 12 to about 20 hours or any amount therebetween, and washing in 0.2×SSC/0.1% SDS at 42° C. for 30 minutes each wash (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C. from about 12-20 hours or any amount therebetween, and washing in 0.1×SSC/0.1% SDS at 68° C. for 30 minutes (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, but not wishing to be limiting, stringent conditions are selected to be about 5° C. lower than the thermal melting point for the specific sequence at a defined ionic strength and pH.

The present invention also includes compositions and methods using plasmid constructs for obtaining the transformed host cell. These include plasmid vectors for transforming a host cell comprising a polynucleotide that encodes a polypeptide having an amino acid sequence selected form the group consisting of SEQ ID NO: 1; SEQ ID NO: 2; SEQ ID NO: 3; SEQ ID NO: 4; SEQ ID NO: 5 and fragments thereof. There is further provided a plasmid vector for transforming a host cell comprising a polynucleotide that encodes a polypeptide having an amino acid sequence selected form the group consisting of SEQ ID NO: I; SEQ ID NO: 2; SEQ ID NO: 3; SEQ ID NO: 4; SEQ ID NO: 5 and fragments thereof, operably linked to a nucleotide sequence capable of directing expression of the polypeptide in the host cell. The plasmid vector may further comprise a selectable, storable or visible marker gene as would be known to one of skill in the art, to facilitate the detection of the transformed host cell.

Plasmid vectors of the invention may comprise any suitable promoter sequence as would be known to one of skill in the art, including constitutive, inducible, tissue specific, developmentally or environmentally induced promoters. For example if the host is a plant, or an algal cell, the cauliflower mosaic virus promoter, CaMV35S, or other constitutive promoter may be used.

The polynucleotide encoding the MEP of the present invention is preferably constructed and ligated into an appropriate plasmid vector containing selection markers, along with a promoter for regulating production of the nucleic acid in a transgenic host cell wherein the promoter is inserted into the plasmid vector upstream from the polynucleotide. The plasmid vector is used to transform the host cell thereby enabling expression of the introduced polynucleotide in the host cell.

As with other compositions of matter described above, preferred embodiments of the plasmid vector of this invention will be those where the host cell transformed by the plasmid vector is edible, or where the polypeptide encoded by the plasmid vector is a mucosal antigen, or more preferably where the antigenic polypeptide encoded by the plasmid vector is capable of eliciting an immune response against an Eimeria parasite, particularly a mucosal immune response, in the native state of the Eimeria parasite or as derived from standard pharmaceutical expression systems, or where the encoded antigenic polypeptide is a chimeric protein.

The MEP of the present invention is preferably produced in plants where at least a portion of the plant is edible. For the purposes of this invention, an edible plant or portion thereof is one that is not toxic when ingested by the animal to be treated with the polypeptide produced in the plant. Thus, for instance, many common food plants will be of particular utility when used in the compositions and methods of the invention. However, no nutritive value need be obtained when ingesting the plants of the invention in order for such a plant to be included within the types of the plants covered by the claimed invention.

The MEP of the present invention may be expressed in plant leaves and the leaves may be harvested, dried to a moisture content of less than 5% and pulverized using standard milling techniques. Leaves from several plants may be bulked and mixed and the lever of expressed recombinant polypeptide determined using standard techniques. The MEP may also be expressed in the seed, root or tuber of the plant and the seed, root, or tuber may be processed as required. For example, the polypeptide may be combined with feed filler and then pressed into a feed pellet, which functions as an edible vaccine.

Plants of particular interest in the methods of the invention include Brassica plants, tobacco plants, and oriental melon plants as described in more detail in the examples below. However, it will be understood by those of skill in the art of plant transformation that a wide variety of plant species, for example but not limited to barley, wheat, maize, soybean, potato, cucumber canola, melon, a grass, and alfalfa are amenable to the methods of the invention. All such species are included within the definitions of the claimed invention including algae, fungi, gymnosperms and both dicotyledonous as well as monocotyledonous angiosperm plants.

The present invention therefore provides a host, for example but not limited to a transgenic plant, or a plant cell comprising a recombinant polynucleotide encoding an MEP comprising two or more than one two proteins or protein fragments that are expressed either in the same parasite but from different life stages, different cellular locations, and for example may comprise but are not limited to, a surface protein or a fragment thereof, a microneme protein or a fragment thereof, a refractile body or a fragment thereof, or the protein or protein fragments may be expressed within different a species, within different parasites, or a combination thereof, wherein the MEP exhibits an antigenic response against proteins from different cellular locations, life stages, species of parasite, or a combination thereof.

The present invention also provides a host for example but not limited to a transgenic plant comprising a recombinant polynucleotide encoding an MEP selected form the group consisting of SEQ ID NO: 1; SEQ ID NO: 2; SEQ ID NO: 3; SEQ ID NO: 4; SEQ ID NO: 5, a fragment thereof, or a sequence that may be similar to any one of SEQ ID NO's:1-5. The present invention also provides a seed, root, or tuber of the transgenic plant comprising a recombinant polynucleotide encoding one or more polypeptides selected form the group consisting of SEQ ID NO: 1; SEQ ID NO: 2; SEQ ID NO: 3; SEQ ID NO: 4; SEQ ID NO: 5, a fragment thereof or a sequence that may be similar to any one of SEQ ID NO's: 1-5.

The transgenic plants that express and accumulate the MEP of the present invention, can be harvested and processed into forms suitable for dietary consumption such as, but not limited to, powders, granules, pellets or liquids.

The recovery of the transgenic MEP from the plant cell or whole plant may be accomplished in several ways. A preferred method of recovery is achieved by obtaining an extract of the plant cell or whole plant or portions thereof. Alternatively, where whole plants are regenerated by the methods of the invention, the recovery step may comprise merely harvesting at least a portion of the transgenic plant such as leaves, roots or seeds. The extract may be further processed using standard purification techniques known to one of skill in the art, for example salt or pH precipitation, chromatography including size exclusion, ion exchange, or affinity chromatography, gel electrophoresis, and the like.

Methods for constructing transgenic plant cells are also provided by the invention comprising the steps of constructing a plasmid vector or a nucleic acid construct by operably linking a polynucleotide encoding the polypeptide of the present invention to a plant-functional promoter capable of directing the expression of the polypeptide in the plant and then transforming a plant cell with the plasmid vector or nucleic acid construct. If so desired, the method may be extended to produce transgenic plants from the transformed cells by including a step of regenerating a transgenic plant from the transgenic plant cell.

As described in the examples below, the methods of the invention by which plants are transformed may utilize plasmid vectors that are binary vectors. Alternatively, the methods of the invention may utilize plasmids that are cointegrate vectors. Non limiting examples for plasmid vectors include pB121. pHS737, pTHK-1, pTHK-2 or pGEX.

A food composition is also provided by the present invention which comprises at least a portion of a transgenic plant capable of being ingested for its nutritional value, said plant comprising a plant expressing a recombinant polypeptide of the present invention. For the purposes of the invention, a plant or portion thereof is considered to have nutritional value when it provides a source of metabolic energy, supplementary or necessary vitamins or co-factors, roughage or otherwise beneficial effect upon ingestion by the subject animal, for example, but not limited to poultry. Thus, where the animal to be treated with the food is an herbivore capable of bacterial-aided digestion of cellulose, such a food might be represented by a transgenic plant leaf or seed.

The present invention provides a bacterium or a yeast cell comprising a recombinant polynucleotide encoding an MEP comprising two or more than one two proteins or protein fragments that are expressed either in the same parasite but from different life stages, different cellular locations, and for example may comprise but are not limited to, a surface protein or a fragment thereof, a microneme protein or a fragment thereof, a refractile body or a fragment thereof, or the protein or protein fragments may be expressed within different a species, within different parasites, or a combination thereof, wherein the MEP exhibits an antigenic response against proteins from different cellular locations, life stages, species of parasite, or a combination thereof. The bacterium or yeast cell may comprise a recombinant polynucleotide encoding one or more polypeptides selected form the group consisting of SEQ ID NO: 1; SEQ ID NO: 2; SEQ ID NO: 3; SEQ ID NO: 4; SEQ ID NO: 5, and a fragment thereof.

It will also be obvious to those skilled in the art that bacterium and yeast cells containing the polypeptide that has been expressed and accumulated in the cells can be harvested and processed into forms suitable for dietary consumption by humans or animals such as, but not limited to, powders, granules, pellets or liquids.

It should be apparent to those skilled in the art that MEPs can so be a valuable tool in diagnostics for Eimeria infection. By combining several antigenic epitopes into a single chimeric protein more than one infectious agent can be identified using a single serological assay. The combination of multiple protein fragments can be used to identify antibodies produced by a mammalian or avian host (e.g. see U.S. Pat. No. 6,929,795, Bedate et al.).

The present invention will be further illustrated in the following Examples, which do not limit the scope of the invention in any way.

### EXAMPLES

### Example 1

### Construction of Multiple Epitope Proteins (MEP)

Five recombinant MEP proteins, MEP1, MEP2, MEP3, MEP4 and MEP5, were constructed by combining a variety of short peptide sub-units identified from the literature, as described herein.

The peptide sub-units were identified from four different species of Eimeria, namely E. tenella, E. acervulina, E. maxima or E. necatrix, at different life stages and cellular locations. Sub-unit peptides were identified from the following proteins: NPmz19 (Tajima, et al, 2003, Avain Dis. 47: 309-318); Mzp5-7, (Binger et al, 1993, Mol. Biochem. Parasitol 61: 179-187); Eamzp35, (Jenkins, 1988, Nucl. Acids Res. 16: 9863); Easz22, (Jenkins et al, 1989, Mol. Biochem. Parasitol. 32: 153-161); Etmic5, (Brown et al, 2000, Mol. Biochem. Parasitol. 107: 91-102); Etmic4, (Tomley et al, 2001, Int. J. Parasitol. 31:1303-1310); Etmic2, (Tomley et al, 1996, Mol. Biochem. Parasitol. 79: 195-206); Em100, (Pasamontes, et al, 1993, Mol. Biochem. Parasitol. 57: 171-174); Etmic1, (Tomley et al, 1991, Mol. Biochem. Parasitol. 49: 277-288); p43, (Laurent et al, 1993, Mol. Biochem. Parasitol. 62: 303-312); 56 KDa, (Belli et at, 2002, Int. J. Parasitol. 32: 1727-1737); 230 KDa, (1992, Mol. Biochem. Parasitol. 51: 251-262); S07. (Liberator et al, 1989, Nucl. Acids Res. 17: 7104); Ea1A, (Vermeulen et al, 1993, FEMS Microbiol. Lett. 110: 223-229); Eta1 A, (Vermeulen et al, 1992), Unpublished.

Each MEP contains a different combination of sub-unit peptides.

MEP1 (SEQ ID NO: 1) comprises antigens from all four groups, including 34 amino acids from a merozoite surface protein from E. necatrix (NPmz19), a 52 amino acid sequence from an E. tenella microneme protein (Etmic1), a 66 amino acid sequence from E. maxima surface protein (56 Kda), and a 67 amino acid sequence from E. acervulina retractile body (Ea1A) as follows:

E. necatrix NPmz19:
(SEQ ID NO: 11)
VLVDEPNVIEVIARVFIRRKILLKNPWTKEEHQVV
E. tenella Etmic1:
(SEQ ID NO: 28)
WSEWSTTCGTATRKRWTECSATCGGGTKERERWTEYSACSRTCGGGIQ
ERKR
E. maxima 56 Kda:
(SEQ ID NO: 29)
SYYNSPYYSYSSYPSYYNYSYPSYSYSSYPSYYRYSSYPYYNYSYPSY
YNYGSYPYYSYSSYPSWY
E. acervulina Et1a:
(SEQ ID NO: 30)
GAGVAGLQAISTAHGLGAQVFGHDVRSATREEVESCGGKF1GLRMGEE
AEVLGGYAREMGDAYQRAQ

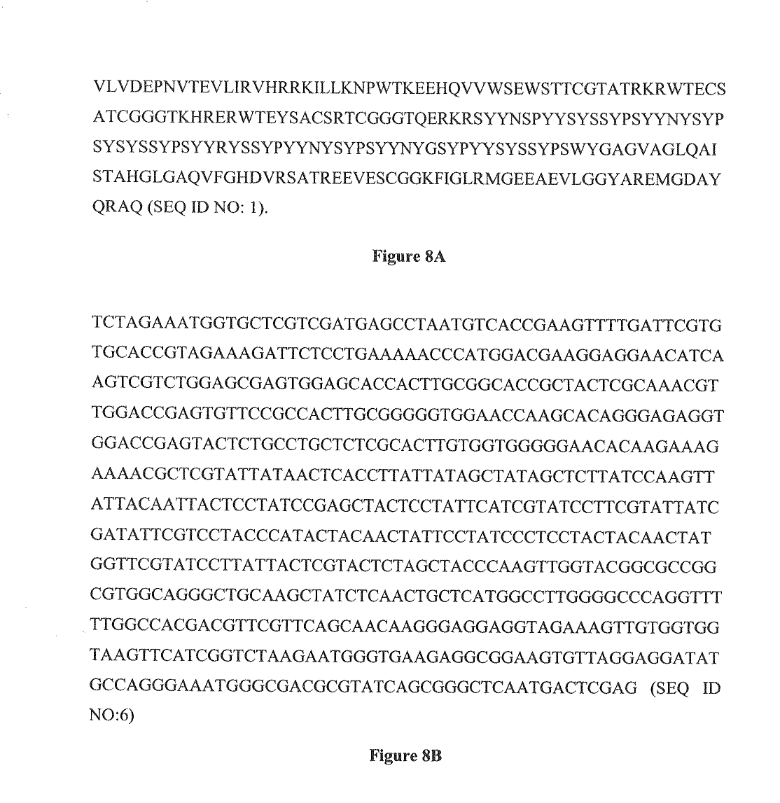

MEP1 comprises the sequence shown in FIG. 8A and SEQ ID NO:1 and is 219 aa, 657 bp.

MEP2 (SEQ ID NO: 2) comprises antigens from proteins expressed at the surface of the parasite, including a 28 amino acid sequence from a surface protein of E. necratrix (NPmz19), a 34 amino acid sequence from the surface of E. acervulina (Easz22), a 34 amino acid sequence from the surface of E. acervulinia (Eamzp35) and a 33 amino acid sequence from E. tenella (Mzp5-7) as follows:

| | |
|---|---|
| E. acervulina EAMZp35: | |
| SPPSTPVSPPSTPVSPPSTPVSPPSTPV | (SEQ ID NO: 12) |
| E. tenella Mzp5-7: | |
| VSVTEPNVDEVLIQIRNKQIFLKNPWTGQEEQVL | (SEQ ID NO: 31) |
| E. necatrix NPmz19: | |
| VLVDEPNVTEVLIRVHRRKILLKNPWTKEEHQVV | (SEQ ID NO: 11) |
| E. acervulina EASZ22: | |
| VVVVVVVGSSMHVVEVRSFGVRRRPSTESRRSS | (SEQ ID NO: 32) |

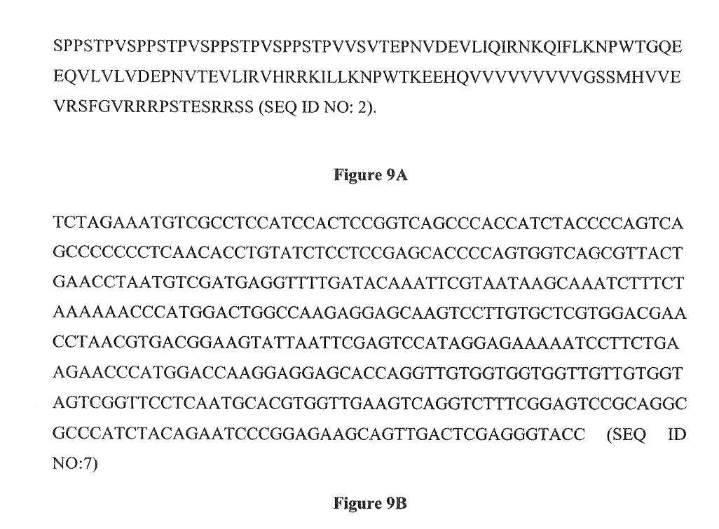

MEP2 comprises the sequence shown in FIG. 9A and SEQ ID NO:2 and is 219 aa or 657 bp.

MEP3 (SEQ ID NO: 3) comprises antigens from proteins expressed in the micronemes and secreted at the surface of the parasites, including a 34 amino acid sequence from an E. necatrix surface protein (NPmz19), a 33 amino acid sequence from an E. acervulina surface protein (Easz22), a 72 amino acid sequence from a protein in the microneme of E. tenella (Etmic1), a 52 amino acid sequence from a protein in the microneme of E. tenella (Etmic2), a 53 amino acid sequence from a microneme of E. tenella (Etmic4), an 82 amino acid sequence from a microneme of E. tenella (Etmic5) and a 23 amino acid sequence from a microneme of E. maxima (Em100).

E. necaTrix NPrnz19:
(SEQ ID NO: 11)
VINDEPNVTEVEIRVHRRKILLKNPWTKEEHQVV
E. acervulina EASZ22:
(SEQ ID NO: 32)
VVVVVVVGSSMHVVEVRSFGVRRRPSTESRRSS
E. tenella Etmic5:
(SEQ ID NO: 33)
SCAVRGSRYGTIPISTQTVDNATLCQQQCQKSSMCEAFSYDIKGKVCY
LHVAYAAKLKRANYNFISGPRQCA
E. tenella Etmic1:
(SEQ ID NO: 28)
WSEWSTTCGTAIRKRWTECSATCGGGTKHRERWTEYSACSRTCGGGTQ
ERKR
E. maxima Em100:
(SEQ ID NO: 34)
WSTTCGSATRQRVWSDWSDCSATCGGGTRYRERWTEFSDCSRVCGGGT
KERRR
E. tenella Etmic4:
(SEQ ID NO: 35)
WTACGDPSEGLRTRTRWSECKNGKQYRGAAGCASVYEVRACSGASDAK
ECWSPWTICR
E. tenella Etmic4-1:
(SEQ ID NO: 36)
DGMQTRDCKSLGVQESRPCSAEGE
E. tenella Etmic2:
(SEQ ID NO: 37)
APKGEGGQEKPSVPLIAVRIHGS

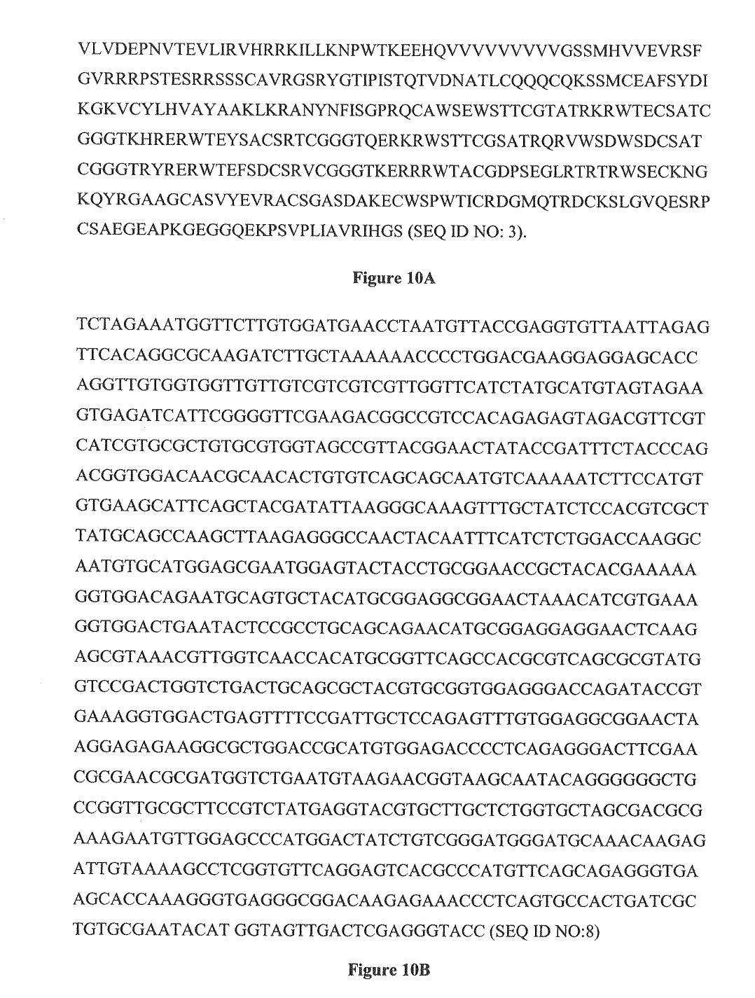

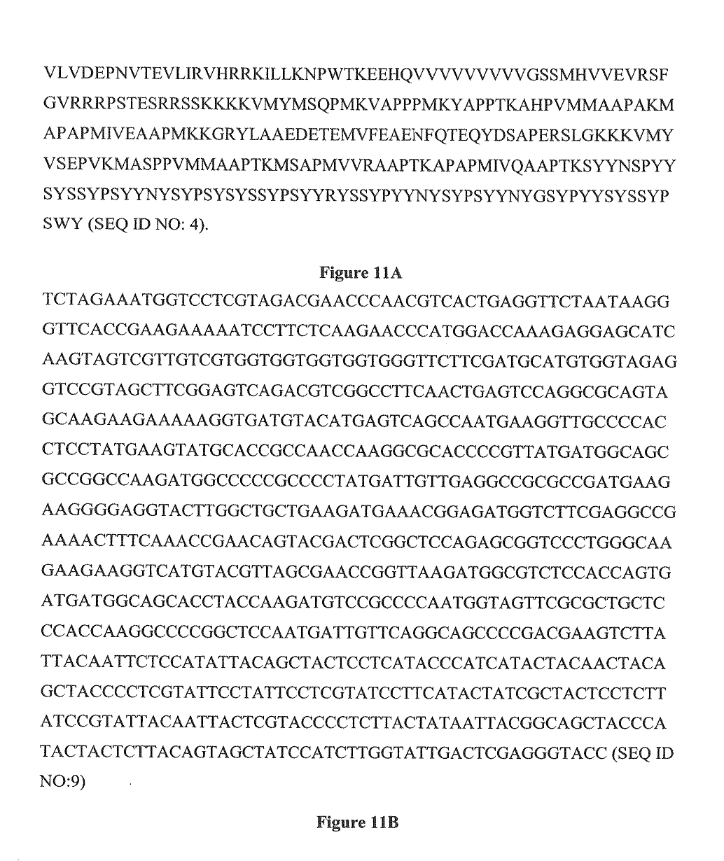

MEP3 comprises the sequence shown in FIG. 10A, and SEQ ID NO:3 and is 349 aa, 1047 bp. Two fragments from the surface group were added to this construct, to create more resistance to more species.

MEP4 (SEQ ID NO: 4) comprises antigens from proteins expressed in the gametocyte, including a 34 amino acid sequence from a surface protein of E. necatrix (NPmz19), a 34 amino acid sequence from a surface protein from E. acervulina (Easz22), a 136 amino acid sequence from a surface protein of E. maxima (230 KDa) and a 66 amino acid sequence from surface protein of E. maxima (56 Kda) as follows:

E. necatrix NPmz19:
(SEQ ID NO: 11)
VLVDEPNVTEVLIRVIARRKILLKNPWTKEEHQVV
E. acervulina EASZ22:
(SEQ ID NO: 32)
VVVVVVVGSSMHVVEVRSFGVRRRPSTESRRSS
E. maxima 230Kda:
(SEQ ID NO: 38)
KKKKVMYMSQPMKVAPPPMKYAPPTKAHPVMMAAPAKMAPAPMIVEA
APMKKGRYLAAEDETEMVFEAENFQTEQYDSAPERSLGKKKVMYVSE
PVKMASPPVMMAAPTKMSAPMVVRAAPTKAPAPMIVQAAPTK
E. maxima 56kda:
(SEQ ID NO: 29)
SYYNSPYYSYSSYPSYYNYSYPSYSYSSYPSYYRYSSYPYYNYSYPS
YYNYGSYPYYSYSSYPSWY.

MEP4 comprises the sequence shown in FIG. 11A and SEQ ID NO:4 and is 269 aa, 807 bp. One fragment from the surface group was added to this construct to create resistance to more species.

MEP5 (SEQ ID NO: 5) comprises antigens from parasites expressed in the retractile body and secreted at the surface of the parasite, including a 34 amino acid sequence from a surface protein of E. necatrix (NPmz19), a 40 amino acid sequence from the retractile body of E. acervulina (p43), a 67 amino acid sequence from the retractile body of E. acervulinia (Ea1 A). a 39 amino acid sequence from a retractile body of E. tenella (Eta1A) and a 40 amino acid sequence from surface antigen of E. acervulinia (SO7) as follows:

E. necatrix NPmz19:
(SEQ ID NO: 11)
VLVDEPNVTEVURVHRRKILLKNPWTKEEHQVV
E. acervulina p43:
(SEQ ID NO: 39)
LLNYHNSQYFGEIKIGTPGRRFVVVFDTGSSNLWVPAAE
E. tenella Et1A:
(SEQ ID NO: 30)
GAGVAGLQAISTAHGLGAQVFGHDVRSATREEVESCGGKFIGLRMGEE
AEVLGGYAREMGDAYQRAQ
E. tenella SO7:
(SEQ ID NO: 30)
QSQVURVSAPSPDEVSRIPRDKVLISYLITSINQQALD
E. acervulina EA1a:
(SEQ ID NO: 41)
DLEQSLEAGKQGAECLIRSSKLALEALLEGARVAATRGLL.

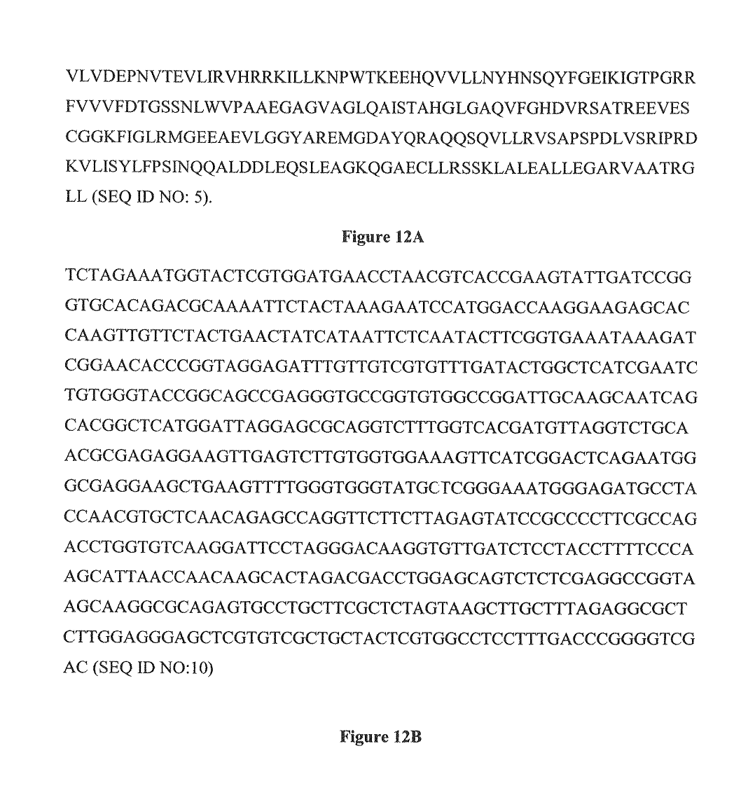

MEP5 comprises the sequence shown in FIG. 12A and SEQ ID NO:5 and is 219 aa, 657 bp. Two fragments from the surface group were added to this construct to create more resistance to more parasite species.

The coding sequences for each of the MEPs were determined and are given as follows:

SEQ ID NO: 6—FIG. 8B; MEP1 (sequence encoding SEQ ID NO: 1)

SEQ ID NO: 7—FIG. 9B; MEP2 (sequence encoding SEQ ID NO: 2)

SEQ ID NO: 8—FIG. 10B; MEP3 (sequence encoding SEQ ID NO: 3)

SEQ ID NO: 9—FIG. 11B; MEP4 (sequence encoding SEQ ID NO: 4)

SEQ ID NO: 10—FIG. 12B; MEP5 (sequence encoding SEQ ID NO: 5)

### Example 2

### Nucleic Acids Sequences Encoding Plant Optimised MEP's

Plant expressible DNA sequences incorporating SEQ ID NO: 6-10 encoding MEP 1-5 respectively, were generated via a computer program devised to select codons for maximum expression in plants. The DNA sequences were constructed essentially as described by Stemmer et al. (Gene 164: 49-53, 1995). Briefly, tens of overlapping oligonucleotides of 40 bases each were synthesized using standard phosphoramidite chemistry. Equal volumes of each oligonucleotide were added to a standard PCR reaction consisting of 10 mM Tris-HCl pH 9.0, 1.5 mM MgCl₂, 50 mM KCl, 0.2 mM each dNTP, 0.1% triton X-100 and 1 u Tag DNA polymerase. The PCR program consisted of 55 cycles of 94° C. for 30 seconds, 52° C. for 30 seconds and 72° C. for 30 seconds. Approximately 2 μl of the resulting mixture was added to a 100 μl PCR reaction mixture as described above and amplified via 30 thermal cycles of 94° C. for 30 seconds, 50° C. for 30 seconds and 72° C. for 30 seconds. The amplified fragment was digested with Bam HI and Xba I and cloned in pUC18 plasmid using standard cloning techniques, Sambrook et al, 2001, Molecular Cloning, Cold Spring Harbour.

Construction of Recombinant Plasmids

Recombinant plasmids were generated using standard techniques as described by Sambrook et al, 2001, Molecular Cloning. A Laboratory manual, Cold Spring Harbor. Briefly, DNA coding sequences for the MEP-proteins were cut from the pUC18 plasmid using the restriction endonucleases XbaI and BamHI. The DNA fragments were purified using agarose gel purification as described by published procedures of Quiagen Corp. Fragments were then ligated into plant binary vectors, using the Xba I and Bam HI cloning sites and T4 DNA Ligase.

### Example 3

### Recombinant MEP Subunit Protein Expression in Bacterial Cells

Transformation of Bacteria Cells

Coding sequences for MEP1, MEP2, MEP3, MEP4 and MEP5 subunit proteins (SEQ ID NO:6-10) respectively were inserted into pGEX vector using the Eco R1 and Xho I restriction sites and transformed into E. coli BL-21 bacterial cell line. The plasmids were confirmed on a 1% agarose gel and working concentrations were confirmed through spectrophotometer analysis. Essentially the transformation protocol for MEP0 (pGEX empty vector) MEP1, MEP2, MEP3, MEP4 and MEP5 vectors into E. coli BL-21 cells is as follows. Approximately 10 ng of plasmid was added to 50 ul E. coli BL-21 cells and incubated on ice for 30 minutes followed by a 30 second heat shock at 42° C. The heat shocked BL-21 cells were put back on ice and 250 ul SOC medium was added and the cells were incubated at 37° C. shaker for 1 hr 225 rpm. The transformed BL-2I cells were plated on Lauria Broth (LB) agar plates with Ampicillin selection and allowed to grow to visible colonies overnight 37° C.

Recombinant Protein Expression

To express the recombinant protein the transformed E. coli BL-21 cells were grown as overnight cultures at 37° C. The following day cultures were diluted 20× and grown to OD 600 between 0.5 and 0.7 after which protein expression was induced with the addition of 1 mM. IPTG for 3 hours. The bacterial cells were lysed and centrifuged for 30 seconds at 12000 rpm and the pellet re-suspended and washed with cold 50 mM Tris(pH7.4). The samples were further centrifuged for 30 seconds at 12000 rpm and again the pellet re-suspended in 100 ul 1×SDS sample buffer, boiled for 5 minutes in a water bath, sonicated for 20 seconds at high power, centrifuged for 15 minutes at 12000 rpm and the proteins transferred to fresh tubes and stored at −20° C.

Assessment of Protein Expression in Bacterial Cells

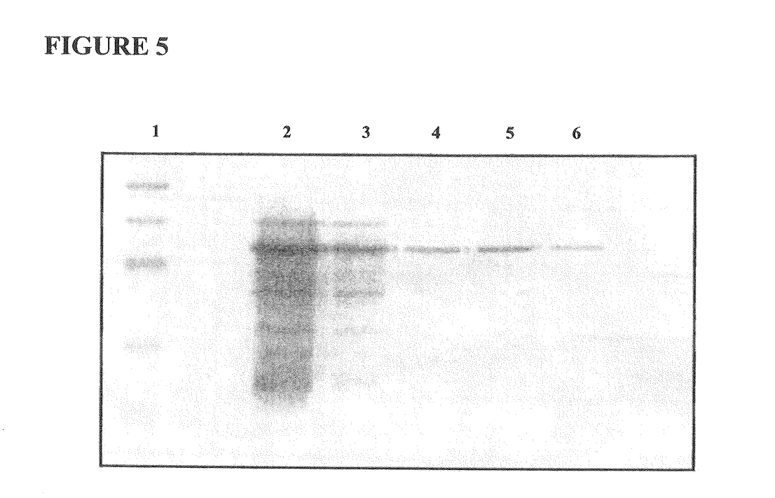

The coding sequence for an MEP subunit protein was cloned into pGEX vector and then transformed into E. coli BL-21 cells as described above. Following transformation the recombinant bacteria were cultured and plated on Lauria Broth Agar and transformants were identified by resistance to Ampicillin (100 ug/ml). Screening of transformed bacteria was done through observation of bacterial plasmid size and through induction of protein expression with comparison to empty vector control transformants. Selected recombinant bacteria cultures were grown to an OD₆₀₀ of 0.5 to 0.7 and expression was induced with addition of IPTG. Optimal conditions were assessed using variations in growth temperature, time of induction and IPTG concentrations. Optimal growth temperature was determined to be 33° C. and optimal IPTG concentration was 0.5 mM. Using these conditions, protein expression was assessed with comparison to non-IPTG induced cultures on every hour and maximal expression was seen at 3-4 hours. Protein extraction was done as follows: bacterial culture samples (1.5 ml) were washed in cold 50 mM tris(pH 7.4). Bacterial pellets were re-suspended in 1×SDS sample buffer (+DTT), boiled for five minutes and sonicated for 30 seconds at high power. Protein in the sample was determined using Bradford analysis and 50-100 ng was loaded onto an SDS-PAGE gel. After completion of the electrophoresis the gel was stained using coomassie staining reagent and analyzed for protein expression of a Glutathion S-Transferase (GST)-fusion protein of known MW. Western analysis was also used to visualize GST-fusion proteins using anti-GST primary antibody and alkaline phosphatase conjugated secondary antibody. FIG. 5 shows a western blot analysis of the GI subunit protein expressed in E. coli BL-21 cells.

MEP subunit protein batch culture and protein isolation

Lysate Preparation of Large Volumes

Stock E. coli containing GST fusion protein of interest was inoculated at the end of the day into 2×500 ml of Lauria Broth containing 50 ug/ml ampicillin and grown at 33° C. overnight. The following morning the OD₆₀₀ was checked to confirm the rate of bacterial growth was between 0.5 and 0.7. If OD was in proper range bacteria were induced with 0.5 mM IPTG and grown for further 3-4 hours. After the induction period the expressing bacteria were split into 4×250 ml centrifuge bottles and centrifuged at 5000×g for 15 minutes. The supernatant was removed and the cell pellet was washed in Iris Borate buffer. The cells were transferred to sterile 15 ml Eppendorf tubes and centrifuged at 2000 rpm at room temperature after which the supernatant was discarded. The cell pellet was then re-suspended in 4 ml PBS/100 ml culture (10 ml PBS/250 ml culture) and two 10 ml volumes were pooled into 50 ml Falcon tubes. Lysozyme (10 mg/ml) was added and the pre-preparation was incubated on ice for 30 minutes. After incubation 10 ml of 0.2% Tween 20, 0.2% Triton x-100 mixture was forcibly added to the mixture to dissolve inclusion bodies and/or precipitated proteins using a 10 ml syringe. Following this addition 200U of DNase 1 and 50 U RNase A were added and the resulting mixture incubated for 10 minutes at ambient temperature with gentle end-to-end mixing. Samples were centrifuged at 3000×g for 30 minutes to remove unwanted cell debris. DTT was added at 1 mM and the supernatant was collected and stored at −80° C. until required for further processing.

GST Mediated Isolation of GST Fusion Proteins

Lysate preparations prepared as described above, were thawed on ice and Glutathione Sepharose 4 Fast Flow (GS4FF) (Amersham) was prepared. The GS4FF was prepared by centrifuging a 50% suspension at 500×g and washing the beads with 10 bed volumes of PBS centrifuging at 500 g and discarding the wash solution in each 2.5 vol. The purification required 2 ml GS4FF bed volume/250 ml culture. After washing was complete the lysate preparations were added to the GS4FF beads and incubated for 45 minutes at room temperature with end-to-end mixing. A sample was removed after binding was completed and washed with another 10 vol. of PBS to remove unbound lysate. After this 2 volumes of elution buffer was added and incubation continued for a further 15 minutes at room temperature with end-to end mixing. Centrifugation of each tube for 10 minutes at 500×g followed and the supernatants were collected and pooled. This process was repeated 4 times. The pooled GST isolates were stored at −80° C. overnight. The following day the samples were freeze dried.

Factor Xa Cleavage of GST Fusion Protein Product

After the pooled protein fraction concentration was determined, Factor Xa was added to cleave the GST moiety from the protein of interest. Factor Xa was added at 10 units per mg eluted protein in the elute and incubated at room temperature for 9 hours. Once digestion was completed the sample was applied to washed and equilibrated GS4FF and incubated for 30 minutes at room temperature. Following this period the supernatant, which contained the protein of interest was collected, the concentration was determined and the supernatant was stored at −80° C. for further usage.

### Example 4

### Recombinant MEP Subunit Protein Expression in Yeast

Transformation of Yeast Cells

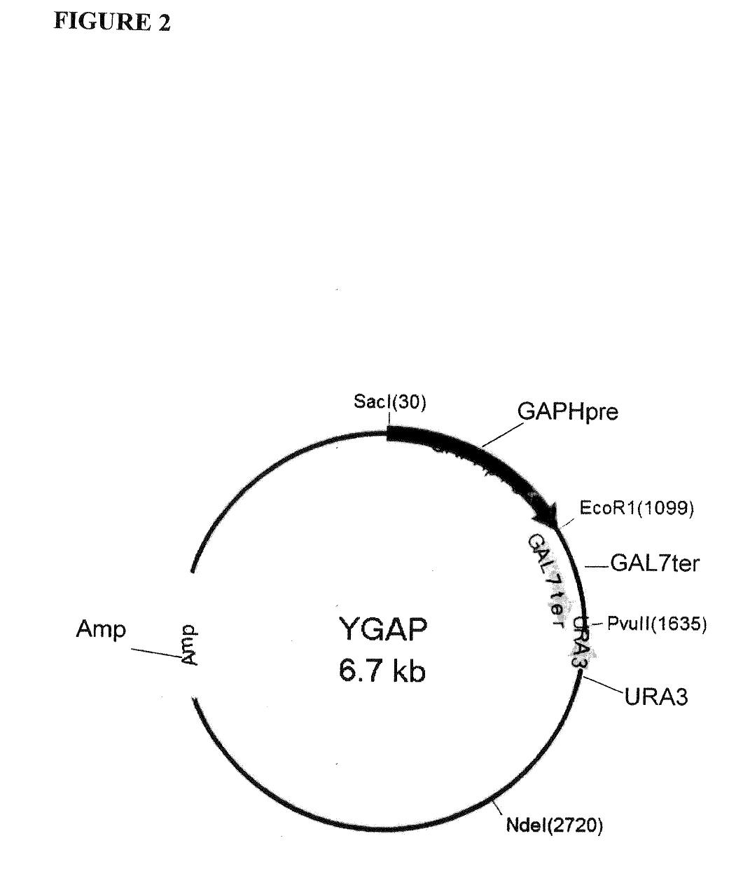

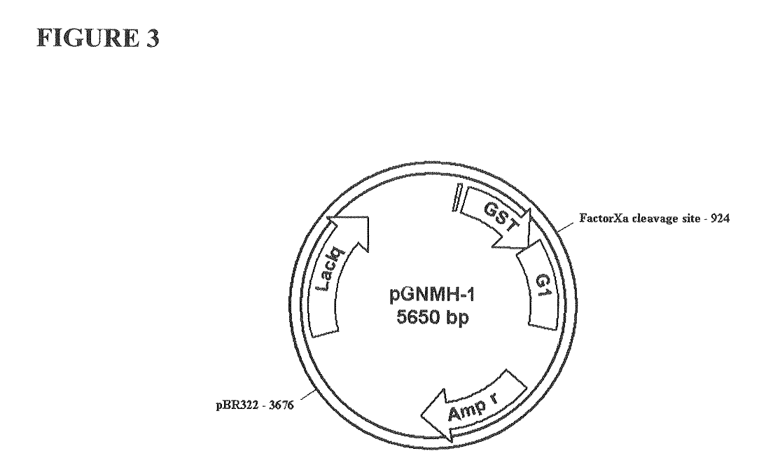

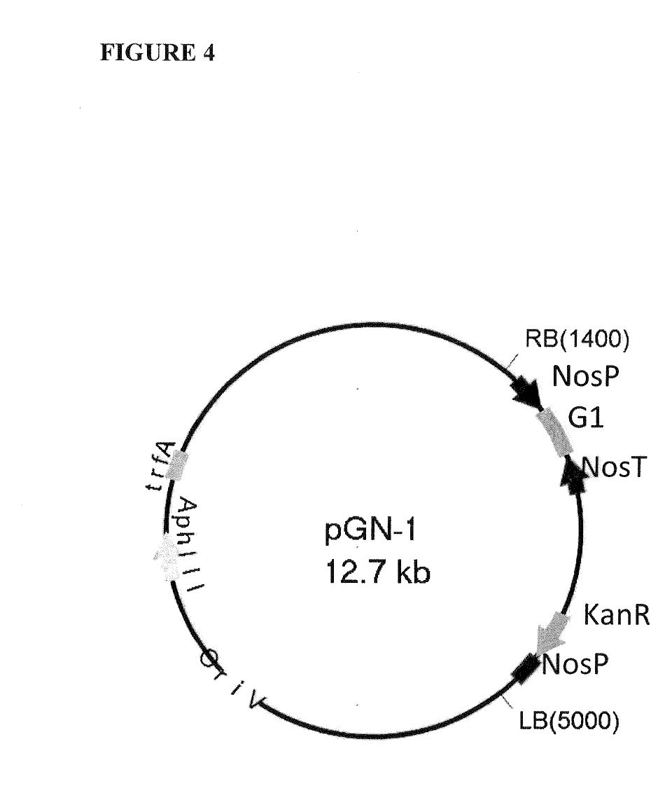

Coding sequences for the MEP subunit proteins (MEP1, MEP2, MEP3, MEP4 and MEP5) were cloned into the yeast expression vector YGAP (see FIG. 2) using standard cloning techniques and using the EcoRI site (Sambrook, et al. 2001, Molecular Cloning. A Laboratory Manual, Cold Spring Harbor).

Recombinant plasmids were introduced into S. cerevisiae as described by Hill et al, 1991, Nucl. Acids Res. 19:5791. Briefly, yeast cells were grown overnight in YPD (1% yeast extract, 2% bacto peptone, 2% dextrose) to an OD₆₀₀ of 0.4. Cells were centrifuged at 3000 rpm for 5 minutes and re-suspended in TE-LIAc buffer (10 mM Tris-HCl, 1 mM EDTA pH 7.5, 100 mM Lithium Acetate). The competent yeast cells were transformed by the addition of the recombinant plasmid, salmon sperm DNA carrier and PEG/TE buffer (40% PEG 3350, 200 mM Lithium Acetate, 20 mM Tris-HCl, 2 mM EDTA, pH 7.5). The reaction mixture was incubated for 30 minutes at 30° C. followed by the addition of 100% DMSO. The yeast cells were heat shocked at 42° C. for 15 minutes and then cooled on ice for 1 minute. Transformed yeast cells were selected by plating the transformation mixture onto selection plates which did not contain uracil (0.67% yeast nitrogen base w/o amino acids, 0.5% caseamino acids, 2% dextrose, 2% agar).

Protein Expression and Isolation from Yeast Cells

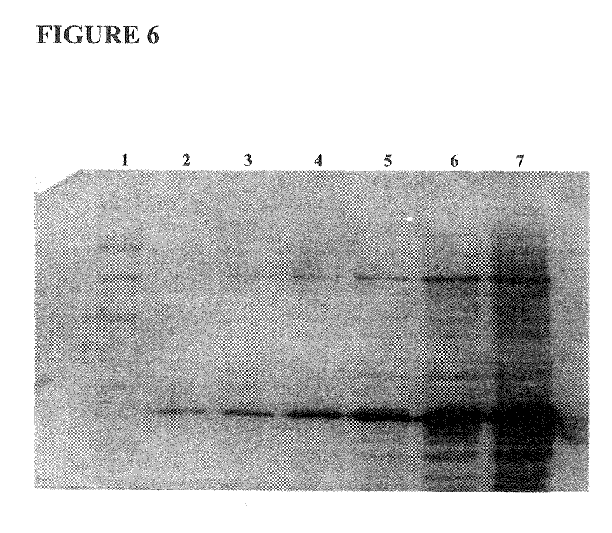

Recombinant MEP subunit proteins were expressed in yeast cells using galactose expression as described by Sambrook et al, 2001, Molecular Cloning. A Laboratory Manual, Cold Spring Harbor. Briefly, yeast cells were grown in expression media containing 1% yeast extract, 2% Bacto peptone, 1% dextrose and 1% galactose. Cells were grown at 30° C. for 30 hours during which time the cells grew rapidly on dextrose. Once the dextrose was consumed the cells converted to galactose utilization and MEP subunit protein expression was initiated. The MEP subunit proteins were isolated from the yeast cells as described by Yaffe et al, 1984, Proc. Natl. Acad. Sci. USA 81; 4819-4823. Essentially, the proteins were isolated as follows; the yeast cells were grown to a cell density of 1×10⁶ and centrifuged at 10,000 g for 15 minutes to pellet the cells. The yeast cells were washed in ice cold water and again centrifuged at 10,000 g for 15 minutes. The washed pellet was re-suspended in 1 ml water containing 100 μg/ml PMSF. Approximately 150 μl of 2N NaOH, 8% 2-ME was added. This was followed by addition of 150 μl of 50% trichloral acetic acid and the resulting mixture was incubation on ice for 10 minutes. The yeast cells were centrifuged at 10,000 g for 2 minutes and the pellet was washed with 1 ml of ice cold acetone. Isolated proteins were suspended in gel buffer (0.2 M Tris-HCl pH 6.8, 6% SDS and 30% glycerol). Complete purification of MEP subunit proteins was conducted using HIS-Select (Sigma) nickel affinity columns following their protocols. Protein in the sample was determined using Bradford analysis and 50-100 ng was loaded onto an SDS-PAGE gel. After completion of the electrophoresis the gel was stained using coomassie staining reagent and analyzed for protein expression of MEP5 protein of known MW. Western analysis was also used to visualize MEP5 using chicken anti-MEP primary antibody and alkaline phosphatase conjugated rabbit anti-chicken secondary antibody. FIG. 6 shows a western blot analysis of the MEP5 subunit protein expressed in yeast cells.

### Example 5

### Recombinant MEP Subunit Protein Expression in Chlamydomonas

Transformation of Chlamydomonas Cells and Protein Expression

Recombinant plasmids used for GST purification in bacteria cells (described above) are also used for transformation of Chlamydomonas cells. A cell wall deficient Chlamydomonas mutant is transformed following the glass bead method of Karen Kindle (1990, Proc. Natl. Acad. Sci. USA 87: 1228-1232). The transformation involves the following: a cell wall deficient Chlamydomona mutant, such as cw10int, is grown to a concentration of 2×10⁶ cells/ml in TAP media. The TAP media is prepared by combining 2.4 g Tris base, 25 ml TAP salts, 0.375 ml Phosphate solution, 1 ml Hunters Trace Elements and 1 ml of Glacial acetic acid to a final volume of 1 L water. Phosphate solution is made using 28.8 g of K₂HPO₄ and 14.4 g KH₂PO₄ in 100 ml water. TAP salts are made by combining 1.5 g NH₄Cl, 4 g MgSO₄.7H₂O and 2 g of CaCl₂.2H₂O in 1 L of water. Hunters trace elements solution is made by combining 22 g of ZnSO₄.7H₂O, 11.4 g of H₃BO₄, 50 g of disodium EDTA, 5.06 g of MnCl₂.4H₂O, 1.61 g of CoCl₂.6H₂O, 1.57 g of CuSO₄.5H₂O, 1.1 g of (NH₄)₆Mo₇O₂₄.4H₂O and 4.99 g of FeSO₄.7H₂O in a final volume of 1 L. Cells are pelleted via centrifugation at 5000 rpm for 5 minutes. The cells are then re-suspended in fresh TAP media to a concentration of 1×10⁸ cells/ml. From this mixture 300 μl is added to a 5 ml test tube containing 0.3 g of 0.4 mm glass beads. Linearised G-plasmid (1 μg) is added to the test tube and the mixture vortexed for 15 seconds. The transformed cells are transferred to 10 ml of TAP medium and the cells amplified overnight by shaking at 100 rpm to allow for recovery and expression of the recombinant MEP protein. The next day the cells are pelleted by centrifugation for 5 minutes at 5000 rpm and re-suspended in 0.5 ml TAP media. The cells are then spread onto selection plates containing TAP with 1.5% agar and an antibiotic. The transformed cells are grown for several days at 22° C. under light of at least 45 μE/m²/s.

Protein Isolation from Transformed Chlamydomonas Cells

Total protein extraction is conducted as described by Perron et al. (1999, EMBO J. 18: 6481-6490). Chlamydomonas cells expressing MEP subunit proteins are pelleted using centrifugation. The cells are then re-suspended in a lysis buffer containing 50 mM Tris pH 6.8, 2% SDS, 10 mM. EDTA, 5 mM 8-amino caproic acid, 1 mM benzamidine HCl, 25 μg/ml pepstatin A and 10 μg/ml leupeptin. Incubation at room temperature for 1 hour is sufficient to disrupt the cells and release the proteins. Subsequent centrifugation results in the pelleted fraction containing membrane bound proteins while the supernatant contains soluble proteins.

For soluble and insoluble fractions, the pellet is re-suspended in lysis buffer without SDS, sonicated on ice and centrifuged at 100 000 g for 30 minutes at 4° C.; the resulting supernatant is the soluble fraction. The pellet is washed with 1 ml of STN solution (0.4 M sucrose, 100 mM Tris pH 8.0, 10 mM NaCl) and centrifuged again at 50 000 g for 15 minutes at 4° C. to remove soluble contaminant proteins. The pellet is re-suspended in lysis buffer and proteins from the insoluble fraction extracted as described for the total protein extract.

### Example 6

### Recombinant MEP Subunit Protein Expression in Plants

Transformation of Canola (Brassica napus)

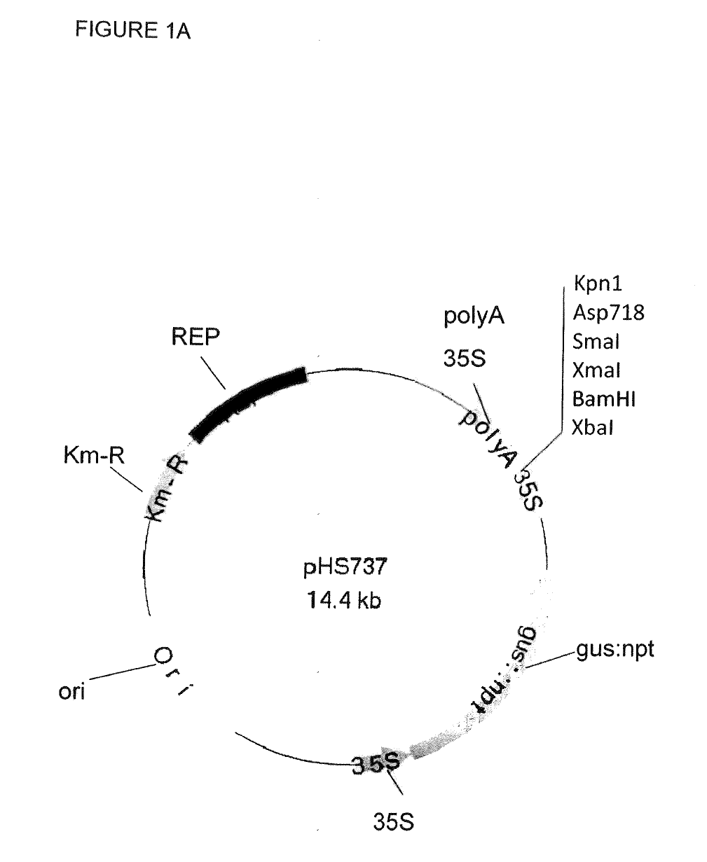

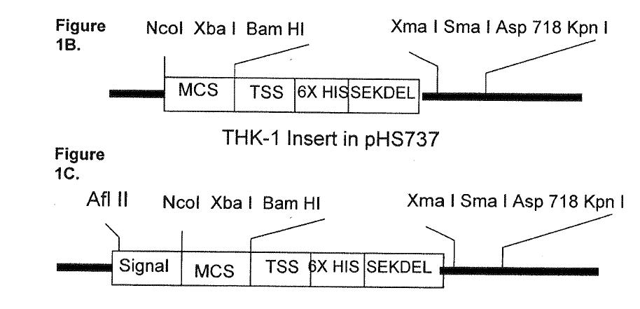

Cotyledons from 4-day old seedlings of Brassica napus cv. Westar that were grown on germination media (2.2 g Murashige and Skoog media, 30 g sucrose pH 5.7 in 1 L water containing 0.8% phytoagar or ½ MMO) were excised and mass dipped in a suspension of A. tumefaciens GV 3101 pMP 90 containing the binary vector pHS737, pTHK-1 or pTHK-2 having the coding sequence for a MEP subunit protein inserted therein (see FIG. 1). After 24 hours of co-cultivation with Agrobacterium at room temperature, cotyledons were transferred into 4° C. for 72 hours. Subsequently, explants were transferred into a solid. Murashige Minimal Organics (MMO) medium containing 300 μg/ml of timentin and 20 μg/ml of kanamycin. All cotyledons were maintained in the growth chamber at 22° C. with 16 hours daylight at 75 μE/m²/s. Shoots regenerated from cotyledonary explants were treated as putative transformants. Effective selection of transformants on kanamycin was achieved. Transformants were grown on kanamycin and timentin rooting media until ready for potting. Plants were transferred to 8 inch pots and grown to maturity in sterilized potting soil.

### Example 7

### Transformation of Cucumis Meld (Oriental Melon

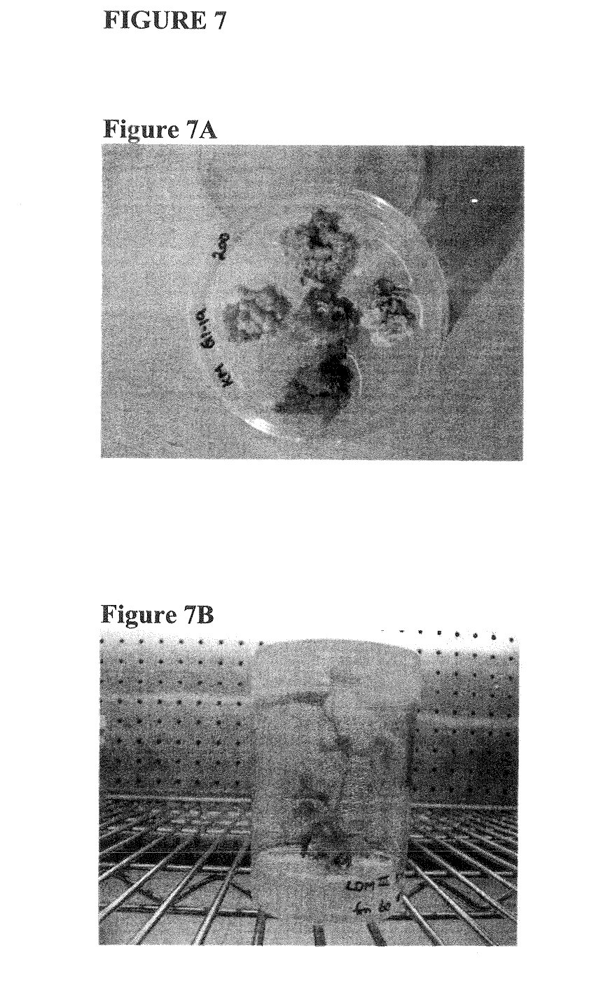

Oriental melons were transformed using a leaf disk procedure modified from Oktem et al, 1999, Tr. J. of Botanty 23: 345-348. Melon seeds were germinated and grown in sunshine potting mix and grown under 16 hour light at 75 μE/m²/s. Leaf disks were harvested from 2-3 month old plants from fully expanded leaves. Leave tissue was surface sterilized by washing in 70% ethanol followed by a 5 minute soaking in 10% bleach. The sterile leaf was washed using multiple changes of sterile water. A leaf disk was isolated using an autoclaved hole punch and it was placed into LDMI medium (4.4 g/l Murashige and Skoog medium, 30 g/l sucrose, 1 μg/ml benzylamino purine, 0.1 μg/ml naphthalene acetic acid, pH 5,7, and 100 μM acetosyringone) containing Agrobacterium tumefacians which had been transformed previously with a binary vector having a coding sequence for a MEP subunit protein inserted therein. The leaf disks were transferred to LDMI agar (LDMI with 0.8% agar which did not contain naphthalene) and the leaf disks were maintained for 3 days at 22° C. using 16 hour daylight at 75 μE/m²/s. The disks were then transferred to LDMII agar (LMDI agar also containing 20 mg/l kanamycin, 300 mg/l timentin) and callus formation occurred over 4 weeks under the above light conditions (see FIG. 7A). Once green shots were formed the callus tissue was removed, residual callus tissue was cut away and it was transferred to LDMII agar for further independent growth. The formation of roots was induced by transferring the shoot tissue to LDMIII media (2.2 g/l Murashige and Skoog medium, 30 g/l sucrose, pH 5.7, 0.8% agar, 20 mg/l kanamycin, 300 mg/l timentin). Roots developed over an incubation period of 3-4 weeks under the light regime described above (see FIG. 78). Once roots develop, the transformed melon plants can be transferred to soil.

### Example 8

### Protein Isolation from Plant Leaf or Seed Tissue

MEP subunit proteins can be isolated from oriental melon or canola leaf tissue using the protocol outlined from the Institute of Molecular Biology, University of Copenhagen online protocols (URL: molbiol.ku.dk). Briefly leaf tissue is harvested using scissors or a scalpel and washed well with water to remove surface debris and any contaminating objects. The tissue is homogenized using WCEB solution (40 mM Tris-HCl pH 7.5, 5 mM MgCl₂, 0.5 M sucrose. 10 mM 2-me, 0.8 mM PMSF) at 4° C. The solution is filtered through cheese cloth to remove debris. After measuring the volume, a 1/10^{th} volume of 5 M NaCl is added and the solution incubated on ice for 30 minutes. The solution is then centrifuged at 20,000 rpm. The supernatant fraction is recovered and the proteins concentrated using ammonium sulphate followed by centrifugation at 10,000 rpm. The resulting protein pellet is re-suspended in a small volume of NEB buffer (40 mM KCl, 25 mM Hepes pH 7.5, 0.1 mM EDTA, 0.8 mM PMSF). Samples are stored at −70° C.

### Example 9

### Purification and Detection of Eimeria proteins

Purification of Eimeria Surface Proteins

Eimeria species oocysts are disrupted using glass bead agitation (MacPherson et al, 1993, Vet. Parasitol. 45: 257-266). A minimum of 100,000 Eimeria oocysts are suspended in sterile water and 0.1 g of 1 mm acid washed beads is added to the suspension. The sample is vortexed at maximum speed for 2 minutes. Cell debris is removed by centrifugation at 3000 rpm. The released sprozoites are used directly for protein isolation.

Cellular proteins are isolated following the procedures described by Hemphill et al, 1997, (Parasitol. 115: 371-380). The sporozoites are concentrated using centrifugation at 10,000 rpm for 10 minutes. The parasite pellet are re-suspended in PBS containing 0.2 mM PMSF. The solution is adjusted to contain 0.75% Triton X-114 and after gentle mixing the parasite proteins are extracted by incubation on ice for 10 minutes. This step is followed by centrifugation at 10,000 g for 30 minutes at 4° C. The Triton X-114 supernatant is collected and incubated for a further 5 minutes at 30° C. and then cooled on ice. The detergent and hydrophobic phases are separated by centrifugation at 1000 g for 5 minutes. The water soluble supernatant is collected and stored at −70° C.

Membrane bound proteins in the pellet fraction are extracted as described in Wessel et al, 1983, (Anal. Biochem. 138: 141-143). A volume of membrane protein fraction is diluted with a 4 fold volume of methanol and mixed using vortexing. A volume of chloroform is added and the sample mixed well by vortexing. To separate the phases, 3 volumes of water are added and again the sample is vortexed. After subsequent centrifugation at 9000 rpm, the upper phase is discarded. An additional 3 fold initial volume of methanol is added to the mixture and the sample is mixed well. The proteins are pelleted by centrifugation at 9000 rpm for 2 minutes. The isolated membrane bound proteins are air dried and stored at −20° C.

Immunological Detection of Eimeria Proteins

For immunological detection of Eimeria proteins, chickens are challenged with purified proteins. Three weeks after inoculation, blood was recovered from the chickens by wing vein puncture. After clotting, cellular debris was removed by centrifuging the blood and the polyclonal antisera was recovered. This provides chicken polyclonal antisera immunologically reactive to the MEP subunit proteins.

Detection of Proteins

Western analysis is performed as described by Sambrook et al, 2001, Molecular Cloning. A Laboratory Manual, Cold Spring Harbor. Briefly, 20 μg of total cellular protein is dissolved in 10 μl SDS sample buffer and the proteins are separated on a 12% SDS PAGE gel. The proteins are transferred to Immobilon-P membranes using standard western blotting techniques (Sambrook et al, 2001, Molecular Cloning. A Laboratory Manual, Cold Spring Harbor). The membrane containing immobilized proteins is incubated in a blocking solution of 5% skim milk powder, 0.05% Tween-20 in PBS for 1 hour at 4° C. Primary anti-parasite chicken antibody is added at 1/1000 dilution and incubated in the same blocking solution for 12 hours at 4° C. The membrane is washed three times with blocking solution and a secondary anti-chicken alkaline phosphatase conjugated antibody is added at 1/12,000 dilution and the membrane incubated at 4° C. for three hours. The excess antibody is washed away with three exchanges of blocking solution. Antigens are detected by incubating in the presence of nitro blue tetrazolium and 5-bromo-4-chloroindolyl phosphate and detecting the presence of a dark blue precipitate.

Western blot of MEP1 protein expressed in and recovered from E. coli is shown in FIG. 5. Western blot of MEP 5 protein expressed in and recovered from yeast, S. cerevisiae, is shown in FIG. 6.

Inoculation of Chickens with Oral Bacterial Vaccines

One day old chickens were obtained and maintained on non-medicated chick starter. Each group consisted of 20 birds chosen at random. At days, 7, 10 or 13 an immunizing dose of approximately 10 ug of immunizing MEP-protein contained within 1 ml of E. coli suspension was administered orally. A separate group or 20 birds received a dose of 5 ug of purified MEP1 protein that was administered twice at day 7 and 13 by intramuscular injection. At day 20, the chickens were given a 0.5 ml oral dose of E. tenella (1×10⁴/bird). Table 1 shows the administration regime for each treatment group.

TABLE 1
Administration regime for each treatment group
of 20 chickens receiving saline or MEP1.

| | | | E. tenella |
|---|---|---|---|
| | | | challenge |
| Group # | Group | Vaccination | (day 20) |
| 1 | Control | Saline | No |
| 2 | Control | Saline | Yes |
| 3 | Vaccine (oral) | 1 time (day 7) | Yes |
| 4 | Vaccine (oral) | 2 times (day 7, 10) | Yes |
| 5 | Vaccine (oral) | 3 times (day 7,10, 13) | Yes |
| | Vaccine (I/M) | 2 times (day 7, 13) | Yes |

Body weights of the chickens were determined at day 7, 20, and 27 after which gross pathology was determined at necropsy after allowing for a week of infection. Birds were sacrificed and the intestines examined for the presence and size of lesions. Lesions were scored using a standard Eimeria scoring 1-4 system. The results are shown in Table 2.

TABLE 2
Average weight gain and Eimeria infection rate of chickens
from each treatment group receiving saline or MEP1.

| Group | Weight gain (g) | Infection rate |
|---|---|---|
| 1. Saline Control | 454.6 | 0% |
| 2. Eimeria control | 437.2 | 56% |
| 3. oral day 7 | 391.9 | 36% |
| 4. oral day 7, 10 | 396.6 | 48% |
| 5. oral day 7, 10, 13 | 358.44 | 44% |
| 6. intramuscular | 352.4 | 24% |

The results indicate that the vaccine, taken orally or intramuscularly, is capable of inducing an immune response against Eimeria parasites in chickens.

### Example 10

### Testing of MEPs vs E. Tenella

MEPs constructed as described in Example 1 were tested using a variety of administration methods, including leg injections, in ovo inoculation or oral gavage with pure MEPs, or in ovo inoculation with heat-killed E. coli expressing MEPs. The results were variable, depending on the dosages administered, environmental factors such as humidity and temperature, as well as scoring methods such as body weight, oocyst shedding or lesion scores. The results of one trial that gave the best results are set forth herein.

The effectiveness of embryo vaccination at day 17 with killed E. coli vaccines containing MEP1, MEP3 or MEP5 proteins against challenge infection with E. tenella using 50 birds per treatment group was evaluated. In this trial, the embryo-vaccinated groups were also compared to a group which was given an oral inoculation of heat-killed E. coli/MEP-1 vaccine.

Materials and Methods:

Embryo Vaccination: Eggs were purchased from a local hatchery. For in ovo immunization, broiler eggs were incubated for 17 days, candled to select fertile eggs at 12 days of embryonation, and injected with MEP-1, MEP-3 and MEP-5 vaccines using the “Intelliject” in ovo injector. Injection was carried out according to the manufacturer's instructions. Briefly, MEP-1, MEP-3 and MEP-5 were diluted in sterile PBS and each egg received 100 ul samples into the amnionic cavity using an 18.0 cm-long 18-guage needle provided by the Avitech (Hebron, Md.).

Production of heat-killed E. coli carrying MEP protein: For large scale cultures, a single colony was inoculated into LB broth with MEP-1/pGEX BL-21, MEP-3/pGEX BL-21, or MEP-5/pGEX BL-21. 0.5 mM of 1PTG was added to 500 ml culture, incubated with continuous gentle shaking (5 hrs, 34° C., 200 rpm). Bacteria were killed by heating the culture 90 min at 80° C. The heat-killed bacteria were harvested and the pellet resuspended with 50 ml 1×PBS.

Vaccination: Fertile eggs were injected with 100 ul of PBS or 100 ul of E. coli vaccine carrying MEP-1, MEP-3 or MEP-5. In this trial, 1×10⁸ colony of killed E. coli MEP-1, MEP-3 and MEP-5 were used.

Chickens: As soon as broiler eggs were hatched they were housed in Petersime Starter brooder units and provided with feed and water ad libitum. Birds were kept in brooder pens in Eimeria-free facility and transferred into large hanging cages in a separate location where they were infected and kept until the end of experimental period.

Parasites: Sporulated oocysts of E. tenella were cleaned by flotation on 5.00% sodium hypochlorite, washed three times with PBS, and viability was enumerated by trypan blue using a hemocytometer. E. tenella strain that was used in this trial was WRL-I. and oocyst number was based on sporulated oocysts.

Eimeria challenge infection: Seven day-old birds were randomly separated into different treatment groups according to their body weights, wing-tagged, and inoculated esophageally with E. tenella (ET) using an oral inoculation needle. Once infected, they were placed into the hanging cages (2 birds/cage).

Body weight gain determination: Body weights of individual birds were determined at days 0 (uninfected), 6, 10 and 18 days post infections with Eimeria.

Lesion Scoring: Ten Birds per Group Tested at Day 5 Post Infection for Lesion Scoring.

Statistical analysis: All values are expressed as the mean±standard error. Mean values for body weight gains was compared among ET infected groups by the Duncan's Multiple Range test following ANOVA using SPSS 15.0 for Windows (SPSS Inc., Chicago, Ill.). Differences among means were considered significant at p<0.05.

### <Experimental Group>

Test groups for body weight

| | | | Immunization | No. of | infection 7^{th} DOB | |
|---|---|---|---|---|---|---|
| Group# | Group Name | Dose | Birds No | Birds for serum | Birds No | Infection |

A. Killed E. coli expressing MEP 1

| | | | | | | | |
|---|---|---|---|---|---|---|---|
| 1 | MEP1-8 | 10⁸/100 | ul | 50 | 6 | 56 | E.t. 50,000 |

B. Killed E. coli expressing MEP 3

| | | | | | | | |
|---|---|---|---|---|---|---|---|
| 2 | MEP3-8 | 10⁸/100 | ul | 50 | 6 | 56 | E.t. 50,000 |

C. Killed E. coli expressing MEP 5

| | | | | | | | |
|---|---|---|---|---|---|---|---|
| 3 | MEP5-8 | 10⁸/100 | ul | 50 | 6 | 56 | E.t. 50,000 |

| | | | |
|---|---|---|---|
| Total | 150 | 18 | 168 |

Control Groups

| | | | Number of | No. of | *No. of | |
|---|---|---|---|---|---|---|
| Group# | Group Name | Dose | Birds | Birds for serum | Birds for serum | Infection |

D. pGEX vector in Killed E. coli

| | | | | | | | |
|---|---|---|---|---|---|---|---|
| 4 | pGEX-8I | 10⁸/100 | ul | 50 | 6 | 56 | E.t. 50,000 |
| 5 | pGEX-8C | 10⁸/100 | ul | 50 | 6 | 56 | none |

E. Non-infected

| | | | | | | | |
|---|---|---|---|---|---|---|---|
| 6 | Null | PBS/100 | ul | 50 | 6 | 56 | none |
| 7 | Infection | PBS/100 | ul | 50 | 6 | 56 | E.t. 50,000 |

F. Fluid levels and oral gavage

| 8 | Fluid | none | | 10 | | none |
|---|---|---|---|---|---|---|
| 9 | MEP1-8 | 10⁸/1 | ml | 50 | 50 | E.T. 50,000 |

| | | | |
|---|---|---|---|
| Total | 260 | 24 | 274 |

TABLE 1
Comparison of weight difference with uninfected control
Weight change day 6

| | Final wt | difference | % change |
|---|---|---|---|
| PBS Uninfected | 384 | 0 | 0 |
| PBS Infected | 349 | 35 | 9.1 |
| MEP1 Oral dose | 361 | 23 | 6.1 |
| MEP1 in ovo | 345 | 39 | 10.1 |
| dose | | | |
| MEP3 in ovo | 346 | 38 | 9.9 |
| dose | | | |
| MEP5 in ovo | 343 | 41 | 10.7 |
| dose | | | |

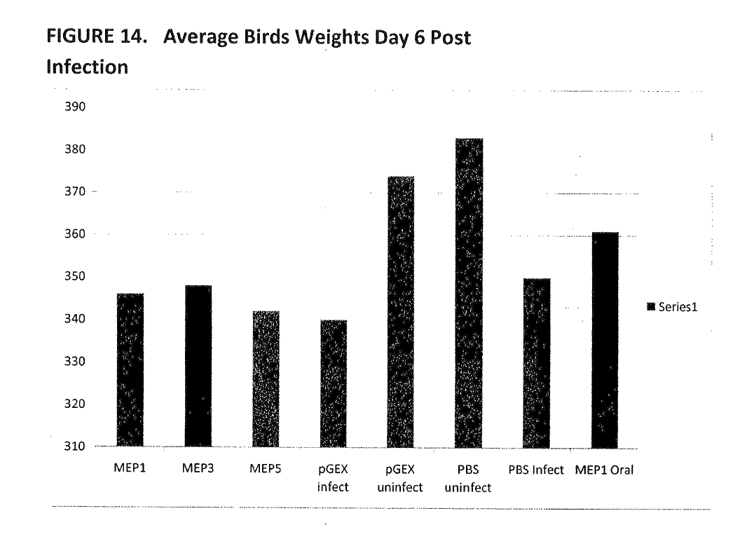

The oral dose administered at day 1 (hatch) had a slight improvement over the infected control group (FIG. 14 and Table 1).

TABLE 2
Comparison of weight differences with uninfected control
Weight change day 10

| | Final wt | difference | % change |
|---|---|---|---|
| PBS Uninfected | 612 | 0 | 0 |
| PBS Infected | 561 | 51 | 8.3 |
| MEP1 Oral dose | 607 | 5 | 0.8 |
| MEP1 in ovo | 577 | 35 | 5.7 |
| dose | | | |
| MEP3 in ovo | 580 | 32 | 5.2 |
| dose | | | |
| MEP5 in ovo | 574 | 38 | 6.2 |
| dose | | | |

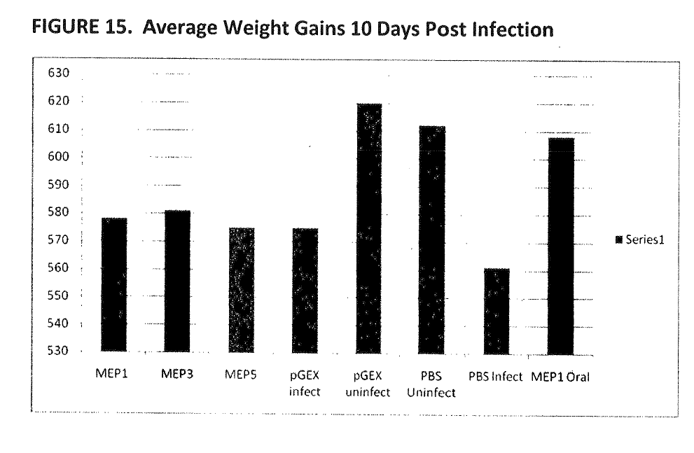

After 10 days the birds' immune systems began to fully fight off parasite infections and the birds that were immunized with the MEP proteins showed improvements. In this trial, MEP3 provided the best protection with the in-ovo vaccination group (FIG. 15 and Table 2). As above, however, the oral gavages with MEP I provided the best protection.

TABLE 3
Comparison of weight difference with uninfected control
Weight change day 18

| | Final wt | difference | % change |
|---|---|---|---|
| PBS Uninfected | 1183.7 | 0 | 0 |
| PBS Infected | 1120.5 | 63.2 | 5.3 |
| MEP1 Oral dose | 1164.5 | 19.2 | 1.6 |
| MEP1 in ovo | 1147 | 36.7 | 3.1 |
| dose | | | |
| MEP3 in ovo | 1154 | 29.7 | 2.5 |
| dose | | | |
| MEP5 in ovo | 1121 | 62.7 | 5.3 |
| dose | | | |

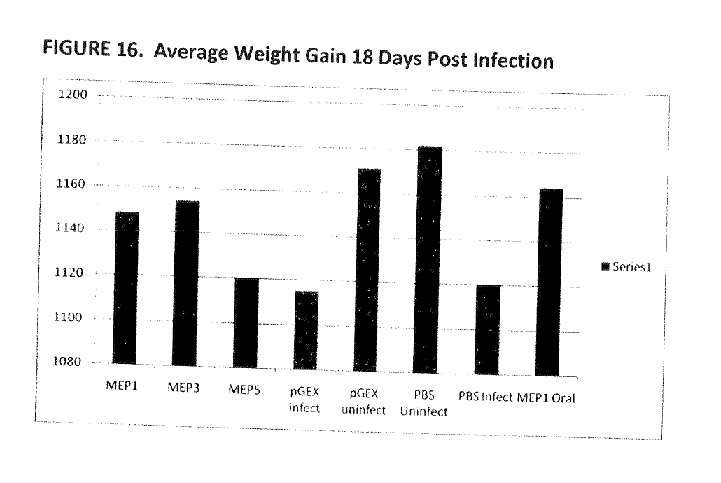

After the birds had been fighting infection for 18 days, the infected birds weights began to catch up with the healthy control birds (FIG. 16 and Table 3). As with the past days, the oral gavages at hatch allowed this group to be only 1.6% behind the control birds weights. The MEP3 vaccination group recovered ½ of the weight difference between the infected control and the healthy control group. MEP3 showed better protection than MEP1 or MEP5 when used in-ovo. Also, the birds followed the typical recover pattern of catching up with the uninfected control over time. At day 6 the infected birds were 9.1% lighter than the control while at day 18 they were 5.3% lighter. The goal with vaccinations is to shorten the time frame needed for infected birds to perform at the same level as healthy birds. MEP3 vaccinations showed this trend where these birds have only 2.5% less body weight.

All citations are hereby incorporated by reference.

The present invention has been described with regard to one or more embodiments. However, it will be apparent to persons skilled in the art that a number of variations and modifications can be made without departing from the scope of the invention as defined in the claims.

What is claimed is:

1. A multi epitope protein (MEP) comprising two or more than two different proteins or different protein fragments, wherein the two or more than two different proteins are selected from the group consisting of SEQ ID N: 13-27 and wherein the two or more than two different protein fragments are selected from the group consisting of SEQ ID NOs: 11, 12, 28-41.

2. The multi epitope protein of claim 1, wherein the two or more than two different protein fragments are selected from the group consisting of SEQ ID NOs 11, 12, 28-41.

3. The multi epitope protein of claim 2, wherein the MEP comprises an amino acid sequence selected from the group consisting of SEQ ID NO: 1, an amino acid sequence having an identity with SEQ ID NO:1 of from about 90% to about 100%, SEQ ID NO: 2, an amino acid having an identity with SEQ ID NO:2 of from about 90% to about 100%, SEQ ID NO: 3, an amino acid having an identity with SEQ ID NO:3 of from about 90% to about 100%, SEQ ID NO: 4, an amino acid having an identity with SEQ ID NO:4 of from about 90% to about 100%, SEQ ID NO: 5, and an amino acid having an identity with SEQ ID NO:5 of from about 90% to about 100%, wherein the identity is determined using BLAST, at default parameters: Program: blastp; Expect 10; filter: default; G=11; E=1; and W=3.

4. A polynucleotide that encodes a multi epitope protein of claim 1.

5. The polynucleotide of claim 4, wherein the MEP comprises an amino acid sequence selected form the group consisting of SEQ ID NO: 1; SEQ ID NO: 2; SEQ ID NO: 3; SEQ ID NO: 4; and SEQ ID NO: 5.

6. A polynucleotide comprising a nucleotide sequence selected from the group consisting of SEQ ID NO: 6; a sequence that hybridizes to a nucleotide sequence of SEQ ID NO: 6; SEQ ID NO: 7; a sequence that hybridizes to a nucleotide sequence of SEQ ID NO: 7; SEQ ID NO: 8; a sequence that hybridizes to a nucleotide sequence of SEQ ID NO: 8; SEQ ID NO: 9; a sequence that hybridizes to a nucleotide sequence of SEQ ID NO: 9; SEQ ID NO: 10; a sequence that hybridizes to a nucleotide sequence of SEQ ID NO: 10, hybridization is in 0.5 M NaHPO₄, 7% sodium dodecyl sulfate (SDS), 1 mM EDTA at 65° C. for about 12 to about 20 hours, and washing in 0.2×SSC/0.1% SDS at 42° C. for 30 minutes each, wherein the polynucleotide encodes a multi epitope protein that exhibits an antigenic response against one or more than one protein obtained from a parasite, one or more than one protein obtained from different species of the parasite, or one or more than one protein obtained from the parasite and one or more second parasite.

7. A nucleic acid construct comprising the polynucleotide of claim 6 operatively linked to an expression control sequence enabling expression of the polynucleotide in a host cell.

8. A host transformed with the polynucleotide of claim 4.

9. A host transformed with the polynucleotide of claim 6.

10. A polypeptide comprising a plurality of peptide sequences obtained from two or more species of Eimeria.

11. The polypeptide of claim 10, wherein the two or more species of Eimeria are selected from the group consisting of E. tenella, E. acervulina, E. maxima or E. necatrix.

12. The polypeptide of claim 10, wherein each of the peptide sequences are from about 20 to about 300 amino acids.

13. A method of immunizing poultry against coccidiosis comprising administering an effective immunizing dose of the multi epitope protein of claim 1 to the poultry.

14. The method of claim 13, wherein the multi epitope protein is administered orally.

15. The method of claim 13, wherein the multi epitope protein is administered by intramuscular injection.

16. The method of claim 13, wherein the multi epitope protein has been expressed in a host cell, and the host cell comprising the expressed multi epitope protein is administered orally to the poultry.

17. The method of claim 16, wherein the host cell is a plant cell, and plant tissue containing the expressed multi epitope protein is administered to the poultry, the plant tissue selected from the group consisting of a leaf, a root, a tuber, a stem, a fruit, a seed, a flower, and an extract thereof.

18. The method of claim 16, wherein the host cell is bacteria.

19. The method of claim 16, wherein the host cell is yeast.

20. The method of claim 16, wherein the host cell is Chlamydomonas.

21. A method of producing a multi epitope protein having an amino acid sequence selected form the group consisting of SEQ ID NO: 1; SEQ ID NO: 2; SEQ ID NO: 3; SEQ ID NO: 4; SEQ ID NO: 5, comprising providing a transgenic host cell comprising a recombinant polynucleotide encoding the multi epitope protein and expressing the multi epitope protein by the host cell.

22. The host of claim 8, wherein the host is a plant.

23. The host of claim 8, wherein the host is a bacterium.

24. The host of claim 8, wherein the host is a yeast.

25. A seed comprising the polynucleotide of claim 4.
